(19) United States
(12) Patent Application Publication
(10) Pub. No.: US 2026/0151478 A1
(43) Pub. Date: Jun. 4, 2026

(54) Single Domain Anti-CD169 Antibodies

(71) Applicant: Stichting Amsterdam UMC, Amsterdam (NL)

(72) Inventors: Johanna Martina Maria Den Haan, Amsterdam (NL); Alsya Jubilly Affandi, Amsterdam (NL); Hendrik Jacobus Brink, Amsterdam (NL); Raimond Heukers, Amsterdam (NL)

(21) Appl. No.: 19/125,482

(22) PCT Filed: Oct. 31, 2023
(86) PCT No.: PCT/NL2023/050572

§ 371 Date: Apr. 29, 2025

(30) Foreign Application Priority Data
Oct. 31, 2022 (EP) 22204818.3

(51) Int. Cl. A61K 39/395; A61K 39/00; C07K 16/28

(52) U.S. Cl. CPC A61K 39/3955; A61K 39/0005; C07K 16/2803; A61K 2039/505; A61K 2039/55588; A61K 2039/6018; C07K 2317/569

(57) ABSTRACT

The invention provides single domain heavy chain variable antibodies that specifically bind CD169. The invention further relates to methods of modulating an immune response in an individual using the single heavy chain variable domain antibody according to the invention, to an immunoreactive molecule and a pharmaceutical composition, comprising the single heavy chain variable domain antibody according to the invention.

### FIELD

The invention relates to the field of immunology. More specifically, the invention relates to single heavy chain variable domain antibodies which bind to CD169. These antibodies are useful in methods for inducing an immune response against an immunoreactive antigen in an individual.

### INTRODUCTION

Monocytes are members of the innate immune system which play a role in the early detection of danger signals, including endogenous danger signals that are released by tissues undergoing stress and exogenous danger signals induced by pathogens. Together with dendritic cells (DCs) they function as antigen-presenting cells (APCs) that activate the adaptive immune responses, including CD4+ and CD8+ T cells (Jakubzick et al., 2017. Nat Rev Immunol 17:349-62). Their potency in the production of pro-and anti-inflammatory cytokines allows them to govern both local as well as systemic immunity.

Monocytes can be broadly categorized into classical (CD14+CD16−), intermediate (CD14+CD16+), and non-classical (CD14−CD16+) populations (Wong et al., 2012. Immunol Res 53:41-57). The distribution and numbers of monocyte subsets can change dramatically under inflammatory conditions, such as during bacterial or virus infection. In addition, monocytes may play an important role in turning off immune reactions and promote tissue regeneration, and are thought to be involved in the development of autoimmune diseases (Ma et al., 2019. Front Immunol 10:1140). In cancer, monocytes may contribute to tumor progression as the major source of tumor-associated macrophages or myeloid-derived suppressor cells with high immune-suppressive activity (Olingy et al., 2019. J Leukoc Biol 106:309-22). However, monocytes have also anti-tumoral roles as they can engulf tumor cells and process them for antigen presentation.

In inflammatory conditions, monocytes frequently show increased expression of CD169, a type-I interferon (IFN-I)-regulated protein. CD169 (Siglec-1, sialoadhesin) is a sialic-acid binding transmembrane receptor that is normally expressed by a subset of macrophages in the spleen and lymph nodes. These macrophages function as gatekeeper of the immune system and the CD169 molecule is involved in pathogen capture and antigen transfer to DCs, leading to T cell activation (Grabowska et al., 2018. Front Immunol 9:2472). Said CD169+ monocytes may represent activated monocytes with enhanced T cell stimulatory capacity.

### BRIEF DESCRIPTION OF THE INVENTION

The invention provides an antibody such as a single heavy chain variable domain antibody, that specifically binds CD169, but does not impede normal functioning of CD169 such as binding of CD169 to gangliosides. In order to select for antibodies that recognize ligand-bound CD169 and/or that do not block normal ligand binding, selections were performed in the presence of sialic acids that can bind to CD169 (Grabowska et al., 2018. Front Immunol 9:2472). Without being bound by theory, the presence of sialic acids in the selection assays may have resulted in a large number of antibodies that specifically bind CD169, but do not impede normal functioning of CD169.

Said antibody binds to an extracellular domain of CD169. Said antibody comprises complementarity-determining regions (CDRs) having amino acid sequences as depicted in Table 1.

Said antibody may be complexed to one or more immunoreactive antigens. Said one or more immunoreactive antigens may be pathogenic antigens. In an embodiment, said one or more immunoreactive antigens are viral and/or microbial antigens. In an embodiment, said one or more immunoreactive antigens are tumor antigens. In an embodiment, said one or more immunoreactive antigens are self-antigens or food or environmental allergens.

An antibody according to the invention may be complexed to a carrier such as a liposome comprising the one or more immunoreactive antigens. As an alternative, or in addition, said antibody may be covalently bound to the one or more immunoreactive antigens.

The invention further provides a method of modulating an immune response in an individual, comprising administering an antibody according to the invention, such as a single heavy chain variable domain antibody, to the individual. Said individual may further be administered an immune modulating molecule such as an adjuvant and/or a cytokine to the individual.

The invention further provides an immunoreactive molecule comprising the antibody according to the invention, such as a single heavy chain variable domain antibody.

The invention further provides a pharmaceutical composition, comprising the antibody according to the invention, such as a single heavy chain variable domain antibody, and a pharmaceutically acceptable carrier.

The invention further provides an antibody according to the invention, such as a single heavy chain variable domain antibody, for use in a method of modulating an immune response in an individual against an immunoreactive antigen, comprising complexing the single heavy chain variable domain antibody to one or more immunoreactive antigens and providing said antibody-antigen complex to the individual. Said antibody-antigen complex is preferably provided to CD169 positive macrophages, monocytes and/or dendritic cells, preferably AXL receptor tyrosine kinase positive dendritic cells, of the individual. Said antibody preferably is a single heavy chain variable domain antibody according to the invention.

### FIGURE LEGENDS

FIG. 1. VHHs binding to (A) BW cells or (B) human CD169-overexpressing BW-Sn cells at different concentrations as measured by flow cytometry. MFI: mean fluorescence intensity.

FIG. 2. VHHs binding to (A) CHO cells or (B) mouse CD169-overexpressing CHO-Sn cells at different concentrations as measured by flow cytometry.

FIG. 3. VHHs binding to (A) human CD169-overexpressing BW-Sn cells or (B) mouse CD169-overexpressing CHO-Sn cells at 500 nM as measured by flow cytometry.

FIG. 4. VHHs binding to recombinant mouse CD169 at different concentrations as measured by ELISA.

FIG. 5. (A) Brief method used on estimating VHH binding regions using multi-step staining protocol with anti-human CD169 commercial antibody (7-239) or CD169-endogenous ligand GM3-liposome on human CD169-overexpressing BW-Sn cells. (B) VHH effect on binding of commercial antibody clone 7-239 on BW-Sn cells and vice versa. (C) VHH effect on binding of GM3-liposome on BW-Sn cells. All measured by flow cytometry.

FIG. 6. (A) Binding of anti-CD169 VHH clone 1B5 or irrelevant VHH control (clone L8CJ3) to monocyte-derived dendritic cells (moDC) at different concentrations. (B) Selected VHHs binding to human blood dendritic cells (DC) subsets, CD169+ Axl DC, compared to conventional DCs cDC1, cDC2, cDC3, and plasmacytoid DC (pDC) at 500 nM. Additional comparisons with human CD169+CD14+ monocytes and CD169-monocytes are included. All measurements were performed by flow cytometry.

FIG. 7. Selected VHHs binding to mouse spleen macrophages (Mac) and dendritic cells (DC) subsets, CD169+ macrophages, red pulp macrophages (RP Mac), conventional DC1 (cDC1), cDC2, and plasmacytoid DCs (pDCs) at 500 nM as measured by flow cytometry.

FIG. 8. (A) Schematic illustration of liposome encapsulating antigen formulated with anti-CD169 VHH clone 1B5 or irrelevant VHH control (clone L8CJ3) using PEG-linker. (B) Binding or uptake of 1B5-liposome or control liposome to human CD169+ moDC at 500 nM and measured by flow cytometry. Anti-CD169 indicates blocking with commercial antibody clone HSn 7D2 to show binding specificity via CD169.

FIG. 9. For antigen presentation assay, 1B5-liposome or control liposome encapsulating gp 100 peptide were taken up by human CD169+ moDC prior to co-culture with gp 100-specific CD8+ T and production of IFNγ was measured by ELISA.

FIG. 10. Tissue distribution of CD169 targeting VHH 1B5 or non-targeting control VHH in WT or CD169 knockout. Data is presented as % injected dose/g ±SD of 4 mice.

FIG. 11. Selected VHHs effect on binding of HL-60 cells to recombinant human CD169 coated on plate as determined by microscopy.

FIG. 12. Selected VHHs effect on binding of FITC-labeled Campylobacter jejuni of different strains (A) GB14, (B) GB23, and (C) GB31 to THP-1 or human CD169-overexpressing THP-1 (TSn) as measured by flow cytometry. (D) Effect of indicated antibodies on HIV transmission from Tsn cells to the HIV-sensitive cell line TZM-bl.

FIG. 13. Immunization with CD169 antibody 1B5 OVA-containing liposomes resulted in increased OVA-specific CD8+ T cell priming. Shown are percentages of H-2Kb/OVA257-264-specific T cells and IFNγ-producing T cells in C57BL/6 mice after vaccination with CD169-targeting and control liposomes.

FIG. 14. CpG linked to anti-CD169 antibody 1B5 stimulates moDC maturation as detected by CD83 expression and IL-6 production.

### DETAILED DESCRIPTION OF THE INVENTION

### Abbreviations

The term “antibody”, as used herein, refers to a proteinaceous molecule belonging to the immunoglobulin class of proteins, containing one or more domains that bind an epitope on an antigen, where such domains are derived from or share sequence homology with the variable region of an antibody. Antibody binding can be expressed in terms of specificity and affinity. The specificity determines which antigen or epitope thereof is specifically bound by the binding domain. Affinity is a measure for the strength of binding to a particular antigen or epitope. Specific binding, or “specifically recognizing” is defined as binding with affinities (KD) of at least 1×10⁻⁵ M, more preferably 1×10⁻⁷ M, more preferably less than 1×10⁻⁹M. An antibody may be a full length antibody such as a monoclonal antibody comprising immunoglobulin heavy and light chain molecules, a single heavy chain variable domain antibody, and variants and derivatives thereof, including chimeric variants of monoclonal and single heavy chain variable domain antibodies.

The term “single heavy chain variable domain antibody” or “VHH”, as used herein, refers to a an antibody comprising only a single heavy chain variable domain and which is devoid of a light chain. Said antibody preferably is of the type that can be found in Camelidae or cartilaginous fish which are naturally devoid of light chains, or a synthetic antibody which can be constructed accordingly.

The term “cluster of differentiation 169 or CD169”, as is used herein, refers to a lectin-like adhesion molecule that binds glycoconjugate ligands on cell surfaces in a sialic acid-dependent manner. Alternative names are Sialic Acid-Binding Immunoglobulin-Like Lectin 1 (SIGLEC-1) and sialoadhesin.

As described herein, the amino acid sequence and structure of a heavy chain variable domain normally is comprised of four framework regions or ‘FR’, which are referred to in the art and herein as ‘Framework region 1’ or ‘FR1’; as ‘Framework region 2’ or ‘FR2’; as ‘Framework region 3’ or ‘FR3’; and as ‘Framework region 4’ or ‘FR4’, respectively; which framework regions are interrupted by three complementary determining regions or ‘CDRs’, which are referred to in the art as ‘Complementarity Determining Region 1’ or ‘CDR1’; as ‘Complementarity Determining Region 2’ or ‘CDR2’; and as ‘Complementarity Determining Region 3’ or ‘CDR3’, respectively. The total number of amino acid residues of a VHH is typically in the range of 110-120, such as 111, 112, 113, 114 or 115 amino acid residues.

For the purpose of this patent application, the position of CDR1, CDR2, and CDR3 regions is determined with the amino acid residue numbering according to Kabat et al., 1987; 1991 (Kabat et al., 1987. NIH Publication no. 165-462; Kabat et al., 1991. NIH Publication no. 91:3242).

The term “binding”, as used herein in the context of binding between an antibody and an epitope, refers to the process of a non-covalent interaction between molecules. Preferably, said binding is specific. The terms ‘specific’ or ‘specificity’ or grammatical variations thereof refer to the number of different types of antigens or their epitopes to which a particular antibody such as a VHH can bind. The specificity of an antibody can be determined based on affinity. A specific antibody preferably has a binding affinity for its specific epitope of less than 10⁻⁷ M, such as less than 10⁻⁸ M, or even lower.

The term “affinity”, as used herein, refers to the strength of a binding reaction between a binding domain of an antibody and an epitope. It is the sum of the attractive and repulsive forces operating between the binding domain and the epitope. The term affinity refers to the apparent binding affinity, which is determined as the equilibrium dissociation constant (Kd).

The term “antigen”, as is used herein, refers to a molecule or part thereof that can bind to an antibody or T-cell receptor. An antigen comprises one or more epitopes that may trigger an immune response such as a T-cell mediated immune response, a B-cell mediated immune response, or a mixture thereof.

The term “epitope”, also termed “antigenic determinant”, as is used herein, refers to a part of an antigen that is recognized by an antibody (B cell epitope) or by a T cell (T cell epitope). The term epitope includes linear epitopes and conformational epitopes, also referred to as continuous and discontinuous epitopes respectively. A conformational epitope is based on 3-D surface features and shape and/or tertiary structure of the antigen. A posttranslational modification, such as phosphorylation, glycosylation, methylation, acetylation and lipidation, may be relevant for an epitope for recognition by a specific antibody, or by a T cell.

The term “vaccine”, as is used herein, refers to one or more antigens that modulate an immune response. In an embodiment, a vaccine may induce or stimulate immunity and hamper the subsequent development of a disease. For example, a classical vaccine such as the Haemophilus influenzae type b vaccine, comprises one or more highly defined antigens that help to protect against a subsequent infection of H. influenzae. A cancer vaccine may induce the immune system to mount an attack against cancer cells in the body, thereby hampering the further development of that cancer. The term vaccine includes a composition comprising one or more antigens and an immune stimulating molecule such as an adjuvant. In an embodiment, a vaccine may be used to induce tolerance, for example against a particular self-antigen or against a particular food or environmental allergen.

The term “allergen”, as is used herein, refers to a substance, such as pollen, that may cause an allergic reaction through an immunoglobulin E (IgE) response in some individuals.

The term “complex” or “complexed”, as is used herein, refers to two or more parts that may form a unit, but which parts are not necessarily covalently linked. For example, a liposome may be regarded as a complex based on at least one lipid layer surrounding an aqueous compartment. An antibody may be attached such as covalently bound to the lipid layer. The aqueous compartment may comprise compounds such as antigens that are encapsulated by the lipid layer but not bound such as covalently bound to the lipid layer.

The term “encapsulated” as is used herein in the phrase encapsulated in a liposome includes reference to the active compound being associated with a liposome such that it is encapsulated in the aqueous interior of a liposome, interspersed within the lipid layer of a liposome, attached to a liposome via a linking molecule that is associated with the liposome, entrapped in a liposome, complexed with a liposome, contained or complexed with a micelle, or otherwise associated with a liposome.

The term “immune modulating molecule”, as is used herein, refers to a molecule that either stimulates an immune response, or that reduces an immune response, for example to induce immune tolerance.

The term “immune stimulating molecule”, as is used herein, refers to a molecule that enhances an immune response such as an adaptive immune response, for example by facilitating the recruitment, activation or maturation of antigen presenting cells (APCs), by increasing antigen uptake by APCs, by assisting in activating CD4+ or CD8+ T cells, or by stimulating the activity of innate immune cells like dendritic cells. Examples of such immune stimulating molecules include adjuvants, cytokines such as interleukins, tumor necrosis factors, chemokines, and interferons, and molecules that induce such immune stimulating molecules.

The term “immune tolerance”, as is used herein, refers to the reduction or prevention of an immune response against a particular antigen or food or environmental allergen. In general, the immune system is tolerant of self-antigens. Tolerance is maintained, for example, by deletion of B cells that produce self-recognizing antibodies, and by circulating regulatory immune cells, including regulatory T cells and monocytes, that turn off an immune response to restore tolerance.

The term “adjuvant”, as is used herein, refers to a molecule that enhances the immune response to an antigen. Examples of adjuvants known in the art include aluminum salts, monophosphoryl lipid A in combination with an aluminum salt (AS04), an oil in water emulsion composed of squalene (MF59), monophosphoryl lipid A and QS-21, a natural compound extracted from the Chilean soapbark tree, combined in a liposomal formulation (AS01B), and an oil-in-water adjuvant emulsion that contains alpha-tocopherol, squalene, polysorbate 80 (AS03), and cytosine phosphoguanine (CpG), a synthetic form of DNA that mimics bacterial and viral genetic material.

The term “cytokine”, as is used herein, refers to a peptide that plays a role as immunomodulating agents. Cytokines include chemokines, interferons, interleukins, lymphokines, and tumor necrosis factors. A cytokine normally acts through a cell surface receptor and is important in the immune system.

The term “or”, as is used herein, is defined as “and/or” unless specified otherwise.

The term “a” or “an”, as used herein, is defined as “at least one” unless specified otherwise. When referring to a noun in the singular, the plural is meant to be included, unless it follows from the context that it should refer to the singular only. For example, the term “nucleic acid molecule”, as is used herein, includes reference to a collection of two or more nucleic acid molecules that, together, encode a protein of the invention.

### Anti-CD169 Antibodies

Described herein are antibodies that specifically bind CD169, but do not impede normal functioning of CD169 such as binding to a normal ligand of CD169, including gangliosides such as monosialodihexosylganglioside (GM3) (Affandi et al., 2020. PNAS 117:27528-27539).

Some of these antibodies define a specific class of antibodies, namely single heavy chain variable domain antibody antibodies or VHH. The heavy chain variable domain antibodies were isolated from llamas that were immunized with whole cells expressing human CD169. The primary injections were performed with CD169-expressing IFNalpha-treated human monocyte-derived dendritic cells (moDCs), followed by boost injections with IFNalpha-treated human moDCs, human CD169-overexpressing THP1, and a final injection with recombinant human CD169.

Immune phage display libraries were generated from these animals 14 days after the last boost injection. Phages were subsequently panned on plates coated with recombinant CD169. Selections generated four 96 well master plates of individual VHH clones that were then subsequently tested for binding to CD169 by flow cytometry and ELISA.

Said anti-CD169 heavy chain variable domain antibody or VHH preferably has a binding affinity of at most 10-6M, more preferred at most 10-7M, more preferred at most 10-8 M, more preferred at most 10-9 M, more preferred at most 10⁻¹⁰ M. Said binding affinity preferably is between 10-6M and 10-11 M.

A VHH according to the invention preferably comprises amino acid sequences GRTFSNYT, GNIFSINT, GSGFSSSA, GLAFSSYA, GRTFSNYL, GRTFSTYG, GMLFSRAT, GRTFGSYA, GLTFSTYN, GRTENRYF, GGTFSSYH, GSFFSIHA, GIFFSNYV, ERTFGSYA, GSIGSINV, and GIVFRIND for CDR1; INWSGERT, ITYAGST, IFSTGST, INSSGGST, ISPSGGAT, ISQSGGRI, ISTGGLT, ISRAGVRT, ISRTGSNT, ITWSGGTT, INWYGGAT, ITDGGTT, IFSTGYT, ISWNGGLT, MRADSST, and VSSGGST for CDR2; and AQAFTSNTVGRSPANYQH, NRKDWTMAGQGET, KISGSDY, AKDPWLLHSDS, AADGARRVWPGQNVHDYDD, AAQKTHSDSIVFTEELSYEN, NTFDGFHP, AGKSASYDDSVYVAPGQFPF, AADRNLGTWPAKVAHEYDY, AADPSSPNWPPVEYEH, AARRGNFSWHVREYDY, NIDIYTRRVGGFTAY, NADTY, AADPNDYSGSYYYTSESALDSSRYAY, NVVSYSFGARSY, and KAAVQYYNIGSPSGV for CDR3, as determined by the ImMunoGeneTics information system, available at imgt.org (Lefranc, 2014. Front Immunol 5:22. DOI: 10.3389/fimmu.2014.00022).

A preferred antibody comprises CDR amino acid sequences GRTFSNYT (CDR1), INWSGERT (CDR2) and AQAFTSNTVGRSPANYQH (CDR3), or derivatives thereof such as at most 5, including 1, 2, 3 and 4, conservative derivatives.

A preferred derivative may comprise alterations of the amino acid sequence of the CDRs to increase their efficiency, affinity and/or physical stability including, for example a conservative derivative. The term “conservative derivative”, as used herein, denotes the replacement of an amino acid residue by another, biologically similar residue. Examples of conservative derivatives include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like. The CDR sequences of a preferred derivate, preferably a conservative derivative, preferably are more than 80% identical, more preferably are more than 90% identical, more preferably are more than 95% identical to the amino acid sequences of CDR1, CDR2 and CDR3 indicated herein above.

A VHH according to the invention preferably comprises amino acid sequences as depicted in Table 3, or a derivative thereof such as a conserved derivative thereof. Said derivative may comprise alterations of the amino acid sequence of the framework regions, for example to increase the physical stability, such as at most 5, including 1, 2, 3 and 4, alterations. Said alterations preferably comprise conservative derivatives as explained herein above.

In addition, said anti-CD169 antibody may comprise a tag at its N-terminus and/or its C-terminus. Said tag may be added to the protein by genetic engineering to allow the antibody to attach to a column that binds specifically to the tag and thereby allowing the tagged antibody to be isolated from impurities. In addition, the tag may be used to detect a tagged antibody, for example in Western blotting experiments or in immunohistochemistry.

Conventional tags for proteins, such as histidine tag, can be used with an affinity column that specifically captures the tagged protein. The tagged protein is subsequently eluted from said column, e.g., a Ni-IDA column for a histidine tag, using a decoupling reagent according to the specific tag (eg., immidazole for histidine tag). Suitable tags include one or more of a His-tag, c-Myc domain, hemagglutinin tag, maltose-binding protein, glutathione-S-transferase, maltose-binding protein, FLAG tag, biotin acceptor peptide, streptavidin-binding peptide and calmodulin-binding peptide, as presented in Chatterjee, 2006. Cur Opin Biotech 17, 353-358). Methods for employing these tags are known in the art and may be used for purifying and/or detection of said VHH antibody.

As an alternative, or in addition, said anti-CD169 antibody may be provided with a tag that allows specific interaction with another protein, for example by employing sortase-mediated transpeptidation (sortagging; Popp et al., 2007. Nat Chem Biol 3:707-708), and/or by provision of a spytag peptide interaction motif that forms an amide bond with its protein partner, SpyCatcher (Zakeri et al., 2012. PNAS USA 109: E690-E697).

A single heavy chain variable domain may be connected to a Fc region, such as a IgG1, IgG2, IgG3, IgG4, IgM, IgD, IgA or IgE Fc region, or functional part thereof via a hinge region. A preferred hinge region is the hinge region of a camelid or human immunoglobulin heavy chain molecules from IgG1, IgG2, IgG3, IgG4, IgM, IgD, IgA or IgE, most preferred from IgG1.

A preferred part of an Fc region is the region comprising the CH2 domain, the CH3 domain, or the CH2 and CH3 domains of IgGs, preferably IgG1 or IgG3, most preferably CH2 and CH3 domains of human IgG1.

### De-Immunization and Humanization of WHHs

Although VHH antibodies hardly induce an immune response after administration to humans, de-immunization and/or humanization may be required for use of the VHH antibodies of the invention in pharmaceutical compositions. De-immunization is a preferred approach to reduce the immunogenicity of the anti-CMV VHH single heavy chain variable domain antibodies according to the invention. It involves the identification of linear T-cell epitopes in the antibody of interest, using bioinformatics, and their subsequent replacement by site-directed mutagenesis to non-immunogenic sequences or, preferably human sequences. Methods for de-immunization are known in the art, for example from WO98/52976.

A further preferred approach to circumvent immunogenicity of antibodies according to the invention when applied to humans involves humanization. Various recombinant DNA-based approaches have been established that are aimed at increasing the content of amino acid residues in antibodies that also occur at the same or similar position in human antibodies while retaining the specificity and affinity of the parental non-human antibody. Most preferred are amino acid residues that occur in antibodies as they are encoded by genomic germ line sequences.

Preferred methods for humanizing antibodies include grafting of CDRs (Queen et al., 1989. PNAS 86:10029; Carter et al., 1992. PNAS 89:4285; resurfacing (Padlan et. al., 1991. Mol Immunol 28:489; superhumanization (Tan et. al., 2002. J Immunol 169:1119), human string content optimization (Lazar et al., 2007. Mol Immunol 44:1986) and humaneering (Almagro et. al., 2008. Frontiers Biosci 13:1619). Further preferred methods are described in the published international applications WO2011080350; WO2014033252 and WO2009004065; and in Qu et al., 1999. Clin. Cancer Res. 5:3095-3100; Ono et al., 1999. Mol. Immunol. 36:387-395; These methods rely on analyses of the antibody structure and sequence comparison of the non-human and human antibodies in order to evaluate the impact of the humanization process into immunogenicity of the final product.

### Methods to Produce an Anti-CD169 Antibody

An antibody as described, for example a single heavy chain variable domain or an antibody comprising a single heavy chain variable domain, may be produced using antibody producing prokaryotic cells or eukaryotic cells, preferably mammalian cells such as CHO cells or HEK cells, or fungi, most preferably filamentous fungi or yeasts such as Saccharomyces cerevisiae or Pichia pastoris, or mouse ascites. An advantage of a eukaryotic production system is that folding of the protein results in proteins that are more suitable for treating a human individual. Moreover, eukaryotic cells often carry out desirable post translational modifications that resemble posttranslational modifications that occur in mammalian cells.

Production of antibodies, especially of single heavy chain variable domain antibodies, in prokaryotic cells, preferably Escherichia coli, may be performed as described in Arbabi-Ghahroudi et al., 2005 (Arbabi-Ghahroudi et al., 2005. Cancer Metastasis Rev 24:501-519). Production of VHHs in bacteria such as E. coli can be performed by secretion of the antibody into the periplasmic space, or by expression in the reducing cytosol. The latter may require refolding of antibody fragments (Arbabi-Ghahroudi et al., 2005. Ibid.).

Production of antibodies in filamentous fungi is preferably performed as described by Joosten et al., 2005 (Joosten et al., 2005. J Biotechnol 120:347-359, which is included herein by reference. A preferred method for producing antibodies in Saccharomyces cerevisiae is according to a method know in the art (van der Laar et al., 2007. Biotech Bioeng 96, 483-494; Frenken et al., 2000. J Biotechnol 78:11-21). Another preferred method of antibody production is by expression in Pichia pastoris as described by Rahbarizadeh et al., 2006. J Mol Immunol 43:426-435.

A further preferred method for production of therapeutic antibody comprises mammalian cells such as fibroblasts, Chinese hamster ovary cells, mouse cells, kidney cells, human retina cells, or derivatives of any of these cells. A preferred cell is a human cell such as, but not limited to, Hek293, PER.C6, and derivatives thereof.

A single heavy chain variable domain antibody may be produced by the provision of a nucleic acid encoding said antibody to a cell of interest. Therefore, provided herein is a nucleic acid encoding an antibody according to the invention. Said nucleic acid, preferably DNA, may be produced by recombinant technologies, including the use of polymerases, restriction enzymes, and ligases, from the constructs encoding the single heavy chain variable domain antibodies, as is known to a skilled person. Alternatively, said nucleic acid is provided by artificial gene synthesis, for example by synthesis of partially or completely overlapping oligonucleotides, or by a combination of organic chemistry and recombinant technologies, as is known to the skilled person. Said nucleic acid is preferably codon-optimised to enhance expression of the antibody in a selected cell or cell line. Further optimization preferably includes removal of cryptic splice sites, removal of cryptic polyA tails and/or removal of sequences that lead to unfavorable folding of the mRNA. The presence of an intron flanked by splice sites may encourage export from the nucleus and thereby enhance production. In addition, the nucleic acid preferably encodes a protein export signal for secretion of the antibody out of the cell into the periplasm of prokaryotes or into the growth medium, allowing efficient purification of the antibody.

Further provided is a vector comprising a nucleic acid encoding an antibody according to the invention. Said vector preferably additionally comprises means for high expression levels such as strong promoters, for example of viral origin (e.g., human cytomegalovirus) or promoters derived from genes that are highly expressed in a cell such as a mammalian cell (Running Deer and Allison, 2004. Biotechnol Prog 20:880-889; US patent No: 5888809). The vectors preferably comprise selection systems such as, for example, expression of glutamine synthetase or expression of dihydrofolate reductase for amplification of the vector in a suitable recipient cell, as is known to the skilled person.

The invention further provides a method for producing an antibody, the method comprising expressing a nucleic acid encoding an antibody of the invention in a relevant cell and recovering the thus produced antibody from the cell. The nucleic acid, preferably a vector comprising the nucleic acid, is preferably provided to a cell by transfection or electroporation. The nucleic acid is either transiently, or, preferably, stably provided to the cell. Methods for transfection or electroporation of cells with a nucleic acid are known to the skilled person.

A cell that expresses high amounts of the antibody may subsequently be selected. This cell is grown, for example in roller bottles, in fed-batch culture or continuous perfusion culture. An intermediate production scale is provided by an expression system comprising disposable bags and which uses wave-induced agitation (Birch and Racher, 2006. Advanced Drug Delivery Reviews 58:671-685). Methods for purification of antibodies are known in the art and are generally based on chromatography, such as protein A affinity and ion exchange, to remove contaminants. In addition to contaminants, it may also be necessary to remove undesirable derivatives of the product itself such as degradation products and aggregates. Suitable purification process steps are provided in Berthold and Walter, 1994. Biologicals 22:135-150.

Further provided is a host cell comprising a nucleic acid or vector that encodes an antibody according to the invention. Said host cell may be grown or stored for future production of an antibody according to the invention.

### Modifications of an Anti-CD169 Antibody

A single heavy chain variable domain antibody according to the invention may be provided as a bi-or multivalent antibody comprising an anti-CD169 single heavy chain variable domain as described. Said bi-or multivalent antibody may be a bispecific or multispecific antibody comprising two or more single heavy chain variable domains. Said single heavy chain variable domains may be the same, or different recognizing the same or different epitopes on a CD169 molecule, or an epitope on a CD169 molecule and an epitope on another molecule.

An anti-CD169 antibody preferably comprises a heavy chain variable domain directed against CD169 as described herein that is complexed to one or more immunoreactive antigens. Said one or more immunoreactive antigens include one or more pathogenic antigens and/or one or more tumor antigens, such as two or more pathogenic antigens and/or tumor antigens, three or more pathogenic antigens and/or tumor antigens, five or more pathogenic antigens and/or tumor antigens, ten or more pathogenic antigens and/or tumor antigens, twenty or more pathogenic antigens and/or tumor antigens, or fifty or more pathogenic antigens and/or tumor antigens. For practical reasons the number of pathogenic antigens and/or tumor antigens may be maximized to a total of hundred, such as a total of fifty, a total of twenty, a total of ten, a total of five, or even a total of two or one.

Said pathogenic antigen may include at least one fungal, viral, protozoan, or microbial antigen, such as an antigen from a Candida species such as C. albicans, C. tropicalis, and C. parapsilosis, Cryptococcus neoformans, an Aspergillus species such as A. fumigatus and A. flavus, and Histoplasma capsulatum, an arthropod-borne (arbo-) virus such as yellow fever virus, dengue virus, and West Nile virus, hepatitis virus such as hepatitis B virus and hepatitis C virus, herpesvirus such as Epstein-Barr virus and cytomegalovirus, norovirus, papillomavirus, parvovirus, polyomavirus, picornavirus, orthomyxovirus, poxvirus such as variola virus, paramyxovirus, retrovirus such as human immunodeficiency virus, rhabdovirus, a Plasmodium species such as P. falciparum and P. virax, a Leishmania species such as L. major, L. infantum, and L. braziliensis, Trypanosoma brucei, Toxoplasma gondii, a Mycobacterium such as M. tuberculosis, M. leprae and M. lepromatosis, a Streptococcus bacterium such as S. suis and S. pyogenes, Shigella, Campylobacter, a Clostridium bacterium such as C. botulinum, C. tetani and C. perfringens, a Corynebacterium such as C. diphtheriae, Yersinia pestis, Treponema pallidum and a Salmonella bacterium such as S. typhi, S. paratyphi, and S. enteritidis.

Said pathogenic antigen may further include an antigen of a prion that causes a transmissible spongiform encephalopathy such as Creutzfeldt-Jakob disease, Gerstmann-Sträussler-Scheinker syndrome, fatal familial insomnia, kuru, and familial spongiform encephalopathy.

Said pathogenic antigens preferably are conserved antigens, meaning that they are shared by different variants of a pathogen, such as between different isolates, preferably between isolates of different clades. The term “conserved antigen”, as is used herein, refers to an antigen that is at least 80% identical, preferably at least 90% identical, more preferably at least 95% identical, more preferably at least 99% identical between different isolates of a pathogen, preferably between isolates of different clades.

Said one or more tumor antigens comprises one or more epitopes specific for or highly expressed in a cancer, including neo-epitopes. A neo-epitope, also termed de novo epitope, refers to an epitope that arises through a non-synonymous alteration in the genome of a tumor cell that change the amino acid coding sequence. Said neoepitopes include frameshift-mutated antigens, and antigens that have arisen because of tumor-specific splice variants, gene fusions, endogenous retroelements and other classes (Smith et al., 2019. Nature Reviews Cancer 19:465-478). Immune recognition of neoepitopes produced by cancer-specific mutations is a key mechanism for the induction of immune-mediated tumor reduction or even tumor rejection.

Said one or more tumor antigens may further include tumor-associated antigens, such as heat shock proteins, alpha-fetoprotein, and carcino-embryonic antigen, that show differences in expression levels in cancers compared with normal cells. Said tumor-associated antigens include Cancer Testis antigens, of which the expression often correlates with tumor progression (Scanlan et al., 2002. Immunol Reviews 188:22-32).

Said one or more immunoreactive antigens may include a self-antigen that may be involved in an autoimmune disease, and/or a food or environmental antigen that may cause an allergic reaction. Said autoimmune disease includes an antigen that is involved in multiple sclerosis, rheumatoid arthritis, systemic lupus erythematosus, and aplastic anemia. Said food or environmental antigen includes gluten, a cause of coeliac disease, dust mite excretion, plant pollen, peanuts, seafood and shellfish, which are the cause of serious allergies in many people.

The one or more immunoreactive antigens to which an anti-CD169 antibody may be complexed may be directly or indirectly coupled to an anti-CD169 antibody according to the invention.

In an embodiment, a single heavy chain variable domain antibody according to the invention may be complexed to a carrier such as a liposome comprising the one or more immunoreactive antigens. Methods for coupling an antibody such as a single heavy chain variable domain anti-CD169 antibody according to the invention to a liposome are known in the art, including covalent and noncovalent approaches. Common coupling techniques have been described in Nobs et al., 2004 (Nobs et al., 2004. J Pharmaceutical Sciences 93:1980-1992) and in Di et al., 2020 (Di et al., 2020. Advanced Drug Delivery Reviews 154-155:151-162).

A liposome is a lipid layered vesicle with an average particle size of from about 0.5 to about 500 nanometer. Preferred liposomes have a particle size (i.e., average diameter) of from about 1 to about 400 nanometer, more preferably from about 2 to about 200 nanometer, most preferably less than about 200 nanometer.

Liposomes may be prepared from a mixture of phospholipids and cholesterol as is known in the art, e.g. Unger et al., 2012 [Unger et al., 2012. J Control Release 160:88-95; Boks et al., 2015. J Control Release 216:37-46].

A liposome can be loaded with hydrophobic and/or hydrophilic molecules such as one or more immunoreactive antigens. To deliver the one or more immunoreactive antigens to a site of action, the lipid liposome can fuse with other bilayers such as the cell membrane, thus delivering the liposome contents. By preparing liposomes in a solution comprising one or more immunoreactive antigens, said one or more immunoreactive antigens may be delivered over the plasma membrane of a CD169-expressing cell such as a monocyte or a macrophage.

A liposome may be positively charged, neutral or negatively charged, preferably positively or negatively charged, more preferably negatively charged. The liposomes may be a single lipid layer or may be multilamellar such as having a lipid bilayer. A suitable liposome in accordance with the invention preferably is a nontoxic liposome such as, for example, those prepared from phospholipids such as phosphatidylcholine and phosphoglycerol, and often comprise cholesterol. The components of the liposome and/or the amount of each component can be varied using methods known in the art and the formulation which has desirable characteristics (e.g., retention of encapsulated active compound until it is phagocytosed) can be empirically determined.

Said liposome may be modified, for example by polyethylene glycol (PEG), termed PEGylation, for example, to avoid phagocytosis. Said modified liposome may circulate for a prolonged period of time in systemic circulation, when compared to a non-modified liposome. Said PEG may be of any size such as between 0.1 and 60 kDa, and may include branched PEG polymers.

In an embodiment, a single heavy chain variable domain antibody according to the invention may be directly bound, such as covalently bound, to the one or more immunoreactive antigens. Said binding of a single heavy chain variable domain antibody according to the invention to the one or more immunoreactive antigens may involve a linking group which provides conformational flexibility so that the single heavy chain variable domains antibody can interact with its epitope. A preferred linker group is a linker polypeptide comprising from 1 to about 60 amino acid residues, preferably from 2 to about 40 amino acid residues, such as about 3 amino acid residues, 4 amino acid residues, 5 amino acid residues, 6 amino acid residues, 7 amino acid residues, 8 amino acid residues, 9 amino acid residues, 10 amino acid residues, 15 amino acid residues, 20 amino acid residues, or 25 amino acid residues. Some preferred examples of such amino acid sequences include Gly-Ser linkers, for example of the type (Glyx Sery)z such as, for example, (Gly4 Ser)3, (Gly4 Ser)7 or (Gly3 Ser2)3, as described in WO 99/42077, and the GS30, GS15, GS9 and GS7 linkers described in, for example, WO 06/040153 and WO 06/122825, as well as hinge-like regions, such as the hinge regions of naturally occurring heavy chain antibodies or similar sequences as described in WO94/04678.

### Utilization of an Anti-CD169 Antibody

The crux of the invention is a single heavy chain variable domain antibody that specifically binds CD169, but does not impede normal functioning of CD169 such as binding of CD169 to gangliosides. Said antibody may find use in the targeted delivery of vaccines, including cancer vaccines and vaccines for infectious diseases, and for the targeted delivery of tolerance-inducing vaccines. For this, the invention provides an antibody according to the invention for use as a medicament.

The single heavy chain variable domain antibodies according to the invention specifically bind CD169 with high affinity in the low micromolar range, and do not block interaction of CD169-expressing cells with other cells of the immune system as is evidenced by binding of CD169 to gangliosides in the presence of said antibodies. A further advantage of said single heavy chain variable domain antibodies is their small size is that they may more easily contact macrophages in the spleen and lymph nodes to induce an immune reaction against the one or more immunoreactive antigens.

Said antibodies may further find use in reducing or even blocking of virus entry into cells, or in reducing or even blocking transmission of viruses. Said virus includes HIV, Ebola, Marburg virus, a paramyxovirus such as Nipah and Hendra paramyxovirus, and SARS-COV-2 to CD169-expressing cells, such as monocytes and macrophages (reviewed in Raïch-Regué et al., 2022. Mol Aspects Med: doi.org/10.1016/j.mam.2022.10111). For this, a single heavy chain variable domain antibody according to the invention may be used for prophylactic administration or therapeutic administration in an individual such as a human individual that is infected with HIV, Ebola, Marburg virus, a paramyxovirus such as Nipah and Hendra paramyxovirus, Ebola, or SARS-COV-2, or is at risk of being infected with such virus. Thus, antibodies according to the invention may be administered to an individual in order to lessen signs and symptoms of infection, especially of a serious or even fatal infection, or may be administered to an individual already evidencing active infection, especially an individual with weakened immunity.

Said antibodies may further find use in the delivery of antiviral/antibiotic therapeutics to macrophages with intracellular reservoir of virus or bacteria such as Streptococcus pneumonia (Ercoli et al., 2018. Nat Microbiol 3:600-610) and Staphylococcus aureus (Lehar et al., 2015. Nature 527:323-328). Said antiviral/antibiotic therapeutics include antisense-mediated silencing, antibiotics, and toxins.

An antibody according to the invention is preferably administered in an effective amount to an individual in need thereof. An effective amount of an antibody of the invention is a dosage large enough to produce the desired effect. A therapeutically effective amount preferably does not cause adverse side effects, such as hyperviscosity syndrome, pulmonary edema, congestive heart failure, and the like. Generally, a therapeutically effective amount may vary with the individual's age, condition, and sex, as well as the extent of the disease and can be determined by one of skill in the art. The dosage may be adjusted by the individual physician or veterinarian in the event of any complication. A therapeutically effective amount may vary from about 0.01 mg/kg to about 500 mg/kg, preferably from about 0.1 mg/kg to about 200 mg/kg, most preferably from about 0.2 mg/kg to about 20 mg/kg, in one or more dose administrations daily, for one or several days.

An antibody according to the invention can be administered by injection or by gradual infusion over time. The administration of antibodies preferably is parenteral such as, for example, intravenous, intraperitoneal, or intramuscular. Preparations for parenteral administration include sterile aqueous or non-aqueous solutions suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

As an alternative, an antibody according to the invention can be administered by inhalation (Parray et al., 2021. Appl Microbiol Biotechnol 105:6315-6332).

The invention further provides a pharmaceutical composition comprising an antibody according to the invention. A pharmaceutical composition preferably comprises a pharmaceutically acceptable carrier. A carrier, as used herein, means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts buffers, stabilizers, solubilizers, and other materials which are well known in the art.

An anti-CD169 antibody according to the invention may further be used for diagnostic applications. An anti-CD169 antibody of the invention may be labeled by a variety of means for use in diagnostic applications. There are many different labels and methods of labeling known to those of ordinary skill in the art. Examples of the types of labels which can be used include enzymes, radioisotopes, fluorescent compounds, colloidal metals, chemiluminescent compounds, infrared dyes, and bioluminescent compounds.

### EXAMPLES

### Example 1

### Materials and Methods

### Generation of CD169-Targeting VHHs

To generate VHHs against CD169, two llamas (SNL216, SNL217) were immunized with CD169-expressing IFNα-treated human monocyte-derived dendritic cells, human CD169-overexpressing human THP1 cells (Rempel et al., 2008. PLOS One 3: e 1967), and recombinant human CD169 (R&D Systems, Minneapolis, MN). Immunizations and preparation of RNA were performed by Eurogentech (Belgium). VHHs were selected by phage display technology using phage display libraries generated from the two immunized llamas using the phagemid vector pQ81 (QVQ BV) for subsequent transformation into E. coli TG1 as previously described (Gangaiah et al., 2022. MicrobiologyOpen: e1270. Phage-display selections were performed in Maxisorp plates coated with either recombinant human CD169 (R&D Systems) or mouse CD169-FC (produced as previously described by Klaas et al. (Klaas et al., 2012. J. Immunol. 189:2414-2422) protein overnight at a concentration of 5 (μg/mL). In order to select for VHHs that recognize ligand-bound CD169 and/or do not block ligand binding, the first round of selections were performed in the presence of sialic acids that can bind to CD169 using 4% Marvel's blocking buffer in PBS (MPBS) (Grabowska et al., 2018. Front Immunol 9:2472). Counter selections were performed using Maxisorp plates coated with human IgG1 to identify non-CD169 targeting clones. The second round of selections was performed using Carbo-Free blocking solution (Vector Laboratories) that contains no sialic acids. In order to identify cross reactive VHHs, output phages from hCD169 coated plates were used for panning on mCD169 coated plates and vice versa. Four 96 well master plates were generated by infecting TG1 cultures with output phages from the various selection rounds resulting in 368 clones for screening and sequencing. A total of 81 family clusters were identified based on 80% CDR-H3 homology and binding performance to human and mouse CD169 based using ELISA and flow cytometry. 1-2 clones were then selected per cluster for VHH production as previously described (Gangaiah et al., 2021, Microbiology 11:1270) and further analysis (see Table 1).

### VHH Sequencing

To determine the diversity of the VHH, the master plates were sequenced by Sanger sequencing (Eurofins). The nucleic acid sequences were automatically analysed and processed into VHH amino acid sequences using the Pipebio Antibody Sequence Analysis platform (https://pipebio.com/). The VHH sequences were then annotated and subsequently clustered using 80% CDR-H3 homology.

### Recombinant CD169 ELISA

Recombinant human or mouse CD169 proteins were coated on Nunc MaxiSorp ELISA plates (Thermo Fisher Scientific) at 4° C. overnight. This was followed by blocking with Carbo-free blocking buffer (Vector labs, SP-5040-125) for 30 minutes at 37° C. Next, incubation with VHHs for 1 hour at room temperature was performed. This was followed by incubation with an anti-myc tag antibody (Cell Signaling, #2276) in Carbo-free blocking buffer for 1 hour at room temperature. This was followed by incubation with a horseradish peroxidase (HRP)-conjugated anti-mouse antibody (ThermoFisher) in Carbo-free blocking buffer for 1 hour at room temperature. After each incubation step above, washing with 0.05% Tween-20/PBS was performed. Next, incubation with TMB as substrate (Sigma-Aldrich) for 15-30 min at room temperature was performed. Reaction was stopped by the addition of H2SO4 and absorbance was measured at 450 nm using microplate spectrophotometer (Bio-Rad).

### Isolation of Human Primary Cells

Peripheral blood mononuclear cells (PBMCs) from heparinized blood were isolated by density gradient centrifugation (Lymphoprep; Axis-Shield PoC AS). Following PBS washes, cells were further processed for flow cytometry, as described below.

### Monocyte-Derived DCs.

Monocytes isolated using Percoll gradient or CD14-magnetic beads (Miltenyi Biotec) were cultured for 5 to 6 d in RPMI 1640 complete medium (Thermo Fisher Scientific) containing 10% fetal calf serum (Biowest), 50 U/mL penicillin, 50 μg/mL streptomycin, and 2 mM glutamine (all from Thermo Fisher Scientific), in the presence of recombinant human IL-4 (500 U/mL) and GM-CSF (800 U/mL; both from Immunotools).

### Isolation of Mouse Primary Cells

Mouse spleens were mechanically dissociated and digested in a mixture of 3 mg/mL lidocaine, 2 WU/mL Liberase TL (Roche, Mannheim) and 50 mg/mL DNase (Roche, Mannheim) for 12 minutes at 37° C., while the mixture was continuously stirred. Next, ice-cold medium (RPMI-1640 (Gibco, Life Technologies) supplemented with 10% fetal calf serum (FCS, Biowest), 10 mM EDTA, 20 mM HEPES and 50 μM 2-mercaptoethanol) was added, after which the digestion continued for 10 minutes at 4° C. Red blood cells were lysed using an ammonium-chloride-potassium lysis buffer and remaining splenocytes were filtered through a 70-100 μm filter. Following PBS washes, cells were further processed for flow cytometry, as described below.

### CD169-Overexpressing Cells

BW-5147 cells overexpressing human CD169 (BW-Sn) and BW-5147 parental cells (BW; Kirchberger et al., 2005. J Immunol 175:1145-52) were maintained in RPMI 1640 (Thermo Fisher Scientific) complete medium, containing 10% fetal calf serum (Biowest), 50 U/ml penicillin, 50 g/ml streptomycin and 2 mM glutamine (all from Thermo Fisher Scientific). CHO cells overexpressing mouse CD169 (CHO-Sn) and CHO parental cells were maintained in RPMI complete medium. CHO-Sn was cultured under selection medium containing G418.

### Flow Cytometry

Cells were incubated with viability dye (Fixable viability dye eFluor 780, eBioscience, or Live Dead Blue, Life Technologies) and human Fc block (BD Biosciences) or mouse Fc block (in house) in PBS prior to cell surface staining for 10 min at 4° C. Next, cells were washed and incubated with (fluorescence-or biotin-conjugated) antibodies or VHHs in 0.5% BSA/PBS for 20 min at 4° C. In some experiments, an additional secondary incubation step with (fluorescence-or biotin-conjugated) antibodies or VHHs was performed. In some experiments, an additional tertiary incubation step with (fluorescence-conjugated) antibodies was performed. For detection of VHH, antibody against Myc-tag (clone 9B11, Cell Signaling) was used. In some experiments, incubation with DiD-containing GM3-liposome was performed. After each incubation step, cells were washed with 0.5% BSA/PBS. Fixation of cells with 2% paraformaldehyde was performed for 10 min at 4° C. as the final step. Cells were stored at 4° C. up to 1 week until acquisition on Attune (Life Technologies), BD LSRFortessa (BD Bioscience), or Aurora spectral flow cytometer (Cytek) and analyzed on FlowJo software (Tree Star).

### Liposome Preparation

Liposomes were prepared from a mixture of phospholipids and cholesterol utilizing the film extrusion method as described previously [Unger et al., 2012. J Control Release 160:88-95; Boks et al., 2015. J Control Release 216:37-46]. In brief, egg phosphatidylcholine (EPC)-35 (Lipoid GmbH): egg phosphatidylglycerol (EPG)-Na (Lipoid GmbH): Cholesterol (Sigma-Aldrich) were mixed at a molar ratio of 3.8:1:2.5. 3 mol % of ganglioside (GM3, Avanti Polar Lipids), or 1 mol % of 1,2-distearoyl-sn-glycero-3-phosphoethanolamine-N-polyethylene glycol 2000 (DSPE-PEG (2000)) Maleimide (DSPE-PEG-MAL, Avanti Polar Lipids), and 0.1 mol % of lipophilic fluorescent tracer DiD (1, 1′-dioctadecyl-3,3,3′,3′-tetramethylindodicarbocyanine, Thermo Fisher Scientific) were added to the mixture. The solvent was evaporated under vacuum on a rotavapor to generate a lipid film and the residual organic solvent was removed by nitrogen flush. The lipid film was then hydrated in HEPES-buffered saline (10 mM HEPES buffer pH 7.4, 0.8% NaCl) with mechanical agitation by rotary-mixing for 20 min until the lipid film was completely resuspended. For antigen-presentation assay, melanoma-associated antigen gp 100 long peptide

(VTHTYLEPGPVTANRQLYPEWTEAQRLD; 3 mg/mL) were encapsulated into the liposomes during the hydration step. Peptides were produced by solid-phase peptide synthesis using Fmoc-chemistry with a Symphony peptide synthesizer (Protein Technologies). The liposomes were sized by sequential extrusion through two stacked polycarbonate filters (400 and 200 nm) with Lipex high-pressure extrusion device (Northern Lipids). Non-incorporated materials were removed in two consecutive steps by sedimentation of the liposomes by ultracentrifugation using at 200,000 g twice. The final resuspension of the liposomes was performed in HEPES buffer at pH 7.4.

### Liposome Binding and Uptake

For binding assay, cells were incubated with ganglioside-liposomes (100 μM) for 45 min at 4° C. for binding or at 37° C. for uptake. In some conditions, cells were pre-incubated with VHHs or commercial antibodies for at least 20 min at 4° C. Cells were then fixed and acquired on flow cytometry as described above.

### Antibody Conjugation

VHH clone 1B5 or irrelevant VHH control (clone L8CJ3) with C-terminal cysteines were reduced by adding 50 mM TCEP (Sigma Aldrich) at a molar ratio of TCEP:VHH of 3:1 for 2 h at room temperature. VHHs were then incubated with liposomes at VHH: DSPE-PEG-MAL ratio of 1:10 at 4° C. overnight in dark. The unreacted maleimide groups on DSPE-PEG-Mal were quenched by an excess of cysteine of 1:10 (relative to the DSPE-PEG-Mal) and incubated for 1 h at room temperature. After quenching, non-conjugated antibody was removed by dialysis using Spectrum Spectra/Por Float-A-Lyzer G2 (100 kDa, Fisher Scientific) with three times buffer (HEPES-buffered saline) exchange and concentrated using Vivaspin tubes (Sartorius, Epsom, UK) with a molecular weight membrane cut-off of 100 kDa.

### Antigen Presentation

IFNα-treated HLA-A2+ moDCs were seeded at a concentration of 20,000 cells per well in U-bottom 96-well plates, incubated with VHH-liposomes encapsulating gp 100 long peptide (3 h, 37° C.), and followed by medium washes. Antigen-loaded moDCs were then co-cultured overnight with gp 100₂₈₀₋₂₈₈ T-cell receptor (TCR) transduced HLA-A2.1 restricted T cell lines, at a ratio of moDC:T cells of 1:5. LPS (10 ng/ml; Sigma-Aldrich) was also added. After 24 h, production of IFNγ in the supernatants of the co-cultures was determined by ELISA (eBioscience).

### VHH Production

Clones were transformed into the E. coli strain BL21. Pre-cultures were prepared by growing bacteria containing VHH in pQ81 (phagemid vector which generates Myc-6×His tagged VHH with an amber stop codon, based on pUR8100 (Lameris et al., 2016. Immunol 149:111-121); QVQ Holding BV) overnight at 37° C. in 8 ml 2× YT medium supplemented with ampicillin (100 μg/ml) and 2% glucose. The next day, pre-cultures were diluted into 800 ml pre-warmed 2-YT medium and supplemented with 100 μg/ml ampicillin and 0.1% glucose. The bacteria were then grown until OD600 of 0.6-0.9 at 37° C. before induction of the VHH expression with 1 mM of IPTG. VHH were expressed for 4 hours at 37° C. and bacteria were then harvested by centrifugation, and bacteria pellets were resuspended into 30 ml PBS and frozen overnight at −20° C.

Frozen bacteria were thawed at room temperature and centrifuged to separate the VHH-containing soluble fraction from cell debris. VHH were purified from the soluble fraction using immobilized metal affinity chromatography (IMAC) using the C-terminal His-tag on the VHH and agarose resin charged with cobalt (Carl Roth GmbH and Co. KG, Karlsruhe, Germany). Bound VHH were then eluted with 150 mM imidazole, which was later removed by repetitive dialysis against PBS. VHH concentration was determined by UV-VIS spectrometry at 280 nm wavelength. In addition, the purity and integrity of 1 μg of purified VHHs was assessed by Coomassie blue staining of a 15% SDS-PAGE gel after electrophoresis.

### Results

### Generation of CD169-Targeting VHHs

To generate VHHs against CD169, we immunized two llamas with CD169-expressing IFNα-treated human moDCs, human CD169-overexpressing human THP1 cells, and recombinant human CD169. Total RNA was isolated to construct phage-display libraries, and target-binding selection was performed using recombinant CD169. Individual clones were then inoculated to generate four 96 well master plates for screening and sequencing. 81 family clusters were identified based on 80% CDR-H3 homology and binding performance to human and mouse CD169 based using ELISA and flow cytometry. 1-2 clones were then selected per clusters for VHH production and further analysis (Table 1).

### Binding of WHHs to CD169

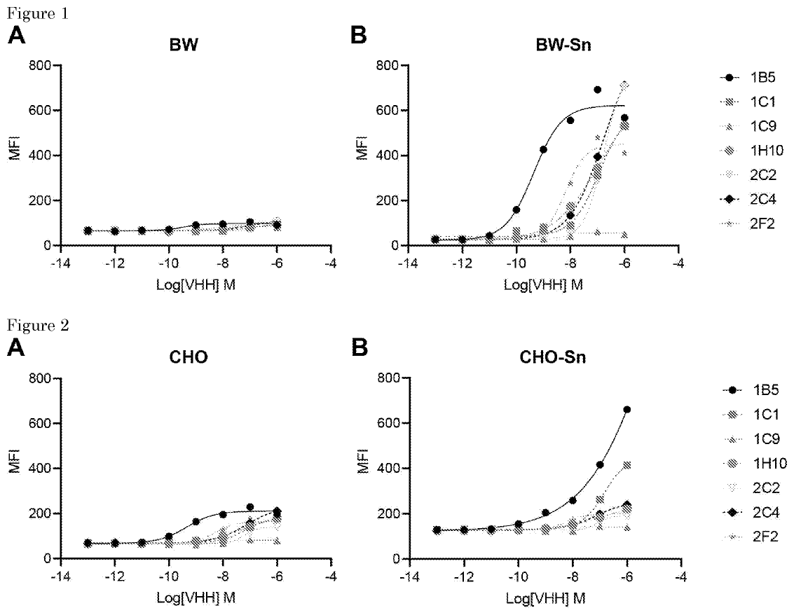

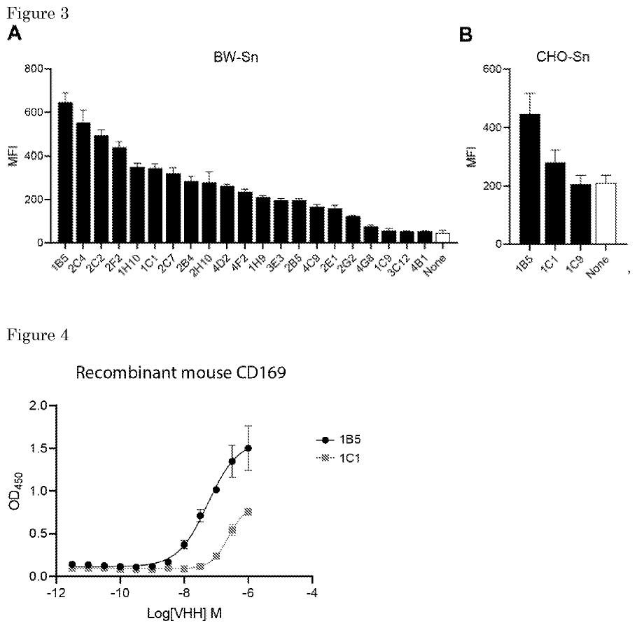

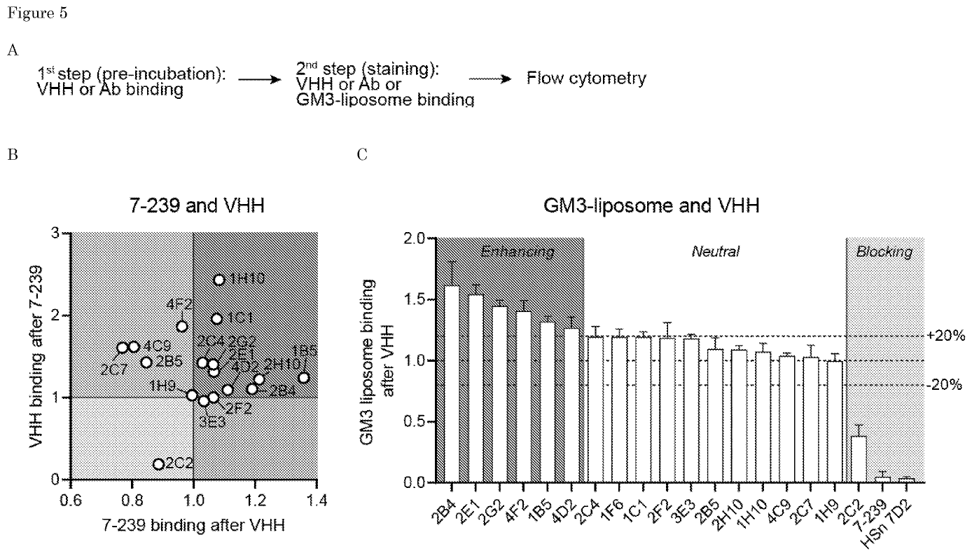

We determined VHHs binding on human CD169-expressing BW-Sn (FIG. 1) and mouse CD169-expressing CHO-Sn (FIG. 2) at different concentrations using flow cytometry. Non CD169-expressing BW and CHO cells were included as negative controls. We verified binding of 16 VHH clones to human CD169, including clone 1B5, 1C1, 1H10, 2C2, 2C4, 2F2, and others. Two of these clones, 1B5 and 1C1, were able to bind to mouse CD169 (FIG. 2). Clone 1C9 was not able to bind to either human and mouse CD169 and this was used as an additional control in further analysis. We further validated VHHs binding at 500 nM to BW-Sn and Cho-Sn in additional experiments (FIG. 3). Furthermore, we calculated EC50 of VHH binding to human CD169 based on binding to BW-Sn (Table 2). Additionally, we performed VHH binding to mouse recombinant CD169 using ELISA (FIG. 4). Therefore, we have generated 16 VHH clones that bind to human CD169 in which two clones cross-react to mouse CD169.

Region Mapping and Competition with Anti-CD169 Commercial Antibody

To further characterize VHHs binding to CD169, we performed sequential staining with an anti-human CD169 commercial antibody clone 7-239 on BW-Sn cells (FIG. 5A). Antibody clone 7-239 recognizes human CD169 and it has been shown to bind to the four N-terminal protein domains. First, we pre-incubated BW-Sn cells with different VHH clones, followed by staining and detection of antibody clone 7-239. Most VHHs did not inhibit antibody clone 7-239 binding to human CD169, in which some VHH clones, including 1B5, seemed to enhance 7-239 binding (FIG. 5B). Only a few VHH clones seemed to inhibit antibody clone 7-239 binding, including clone 2C2. Next, we pre-incubated BW-Sn cells with antibody clone 7-239, followed by staining and detection of VHHs. Almost all VHHs were not affected by pre-incubation with antibody clone 7-239, except for clone 2C2, in which its binding was almost completely suppressed by 7-239. These data indicate that these VHHs differentially affect antibody clone 7-239 binding to CD169 and vice versa, whereas only clone 2C2 share similar or epitope in close proximity to clone 7-239.

VHHs do not Interfere with CD169 Endogenous Ligand Binding

One of the endogenous ligands for CD169 is the sialic acid-containing glycosphingolipid GM3 ganglioside. To investigate whether VHHs binding to CD169 affect its ligand binding capacity, we incorporated GM3 in liposome and performed a binding experiment with BW-Sn. While most VHHs did not negatively affect GM3-liposome binding to BW-Sn, only clone 2C2 inhibited (FIG. 5C). Interestingly, a few VHH clones, including 1B5, enhanced GM3-liposome binding. This suggests that, with the exception of 2C2, the binding of VHHs to CD169 did not interfere with CD169 ligand binding.

### VHHs Binding to CD169-Expressing Primary Cells

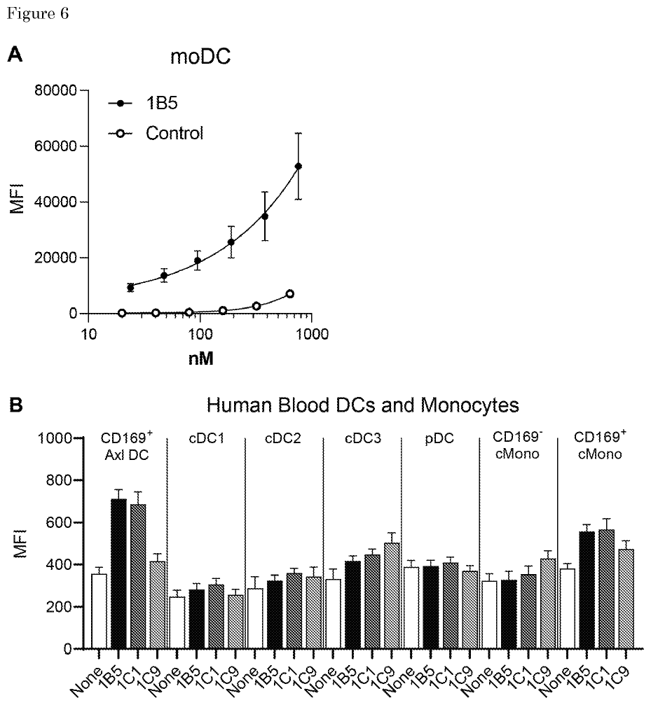

We next validated VHHs binding to CD169-expressing human and mouse primary cells. First, we tested the binding of VHH clone 1B5 to IFNalpha-treated human moDCs at different concentrations (FIG. 6A). Indeed, we saw VHH clone 1B5 binding to IFNalpha-treated human moDCs in a concentration-dependent manner, whereas minimum binding was observed in an irrelevant VHH control. In human blood, CD169 is primarily expressed by human Axl+ DCs, and to a lesser extent, by CD14+ monocytes. We isolated human PBMCs and determined the binding of VHHs binding using flow cytometry. VHH clone 1B5 and 1C1 were able to bind to Axl+ DCs (FIG. 6B), but not to other DC subsets. Additionally, VHH clone 1B5 and 1C1 could also bind to CD169+ monocytes.

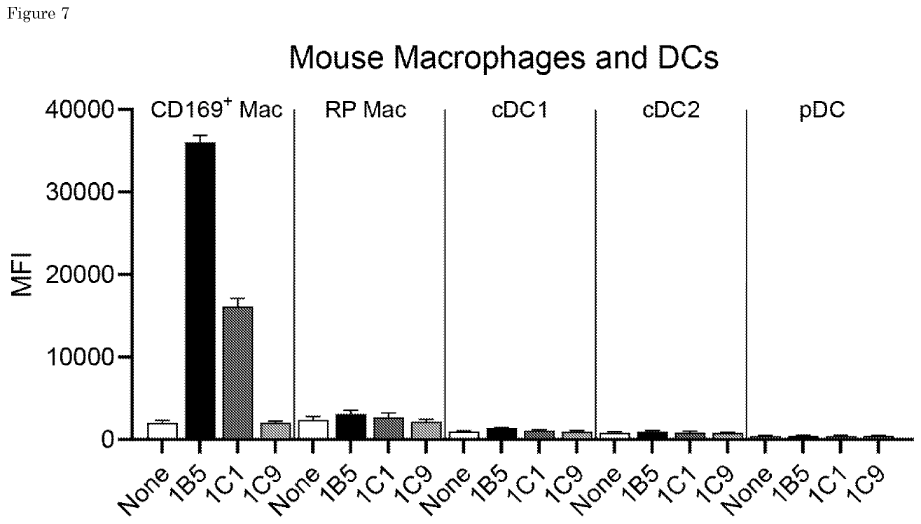

In mice, CD169 is highly expressed by a subset of macrophages called CD169+ macrophages in spleen and lymph node. We isolated and mechanically and enzymatically digested mouse spleens and determined VHHs binding using flow cytometry. VHH clone 1B5, and to a lesser extent clone 1C1, were able to bind to CD169+ macrophages (FIG. 7). Therefore, these VHHs were able to bind to CD169 expressed on human and mouse primary cells.

VHH-Liposome Targeting CD169 Bind to IFNalpha-Treated Human moDC and Deliver Antigen for T Cell Activation

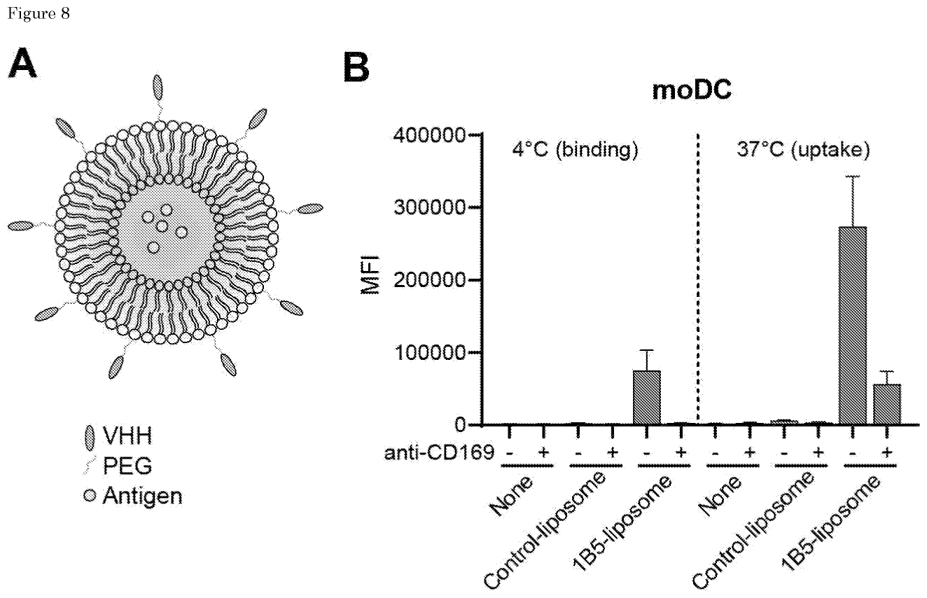

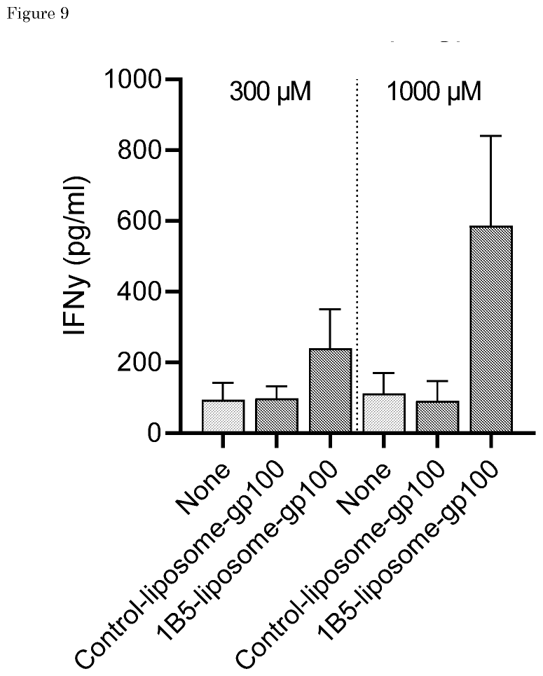

To investigate whether VHH targeting to CD169 could be used for targeted delivery of vaccines, we incorporated VHH clone 1B5 into PEG-liposomes (1B5-liposome) using a maleimide linker (FIG. 8A). We also encapsulated melanoma-associated gp 100 tumor antigen in the VHH-liposome. First, we tested the targeting capacity of 1B5-liposome on IFNalpha-treated human moDCs. We observed that 1B5-liposome could bind and be taken up by IFNalpha-treated human moDCs at 4° C. and 37° C., respectively (FIG. 8B). The 1B5-liposome binding and uptake could be blocked using a commercial anti-CD169 antibody. Control-liposome, containing irrelevant VHH, did not show any binding or uptake in any condition. After incubation with VHH-liposome containing gp 100 tumor antigen (1B5-liposome-gp100), we co-cultured IFNalpha-treated human moDCs with antigen-specific CD8+ T cells for gp 100. We showed that incubation with 1B5-liposome-gp 100 led to IFN-gamma production by T cells, whereas the control-liposome-gp 100 did not (FIG. 9). This indicates that 1B5-liposome can deliver antigen to CD169+ APCs for antigen-specific T cell activation.

### Example 2 PET Imaging

### Materials and Methods

CD169 targeting VHH clone 1B5 and a non-targeting control VHH were both produced containing a free thiol on their C-terminus to facilitate radiolabeling with zirconium [⁸⁹Zr]. VHH's were first reduced with 2 molar equivalents of (tris(2-carboxyethyl)phosphine (TCEP) for 2 hours at 37° C. before being modified using deferoxamine (DFO*)-maleimide at 5 molar equivalents for 60 minutes at 4° C. Afterwards, zirconium labelling was performed at a pH of 7.0 in a thermomixer shaking at 550 rpm at room temperature. Radiochemical purity was subsequently checked by instant thin-layer chromatography (ITLC) and again after 24 hours. Four 6C57BL/6 or CD169 knockout mice were injected with 180 μL of VHH with 3 MBq via a cannula in the tail vein in a PET/CT scanner. Tissue distribution was determined after 1 hour and 7 minutes.

### Results

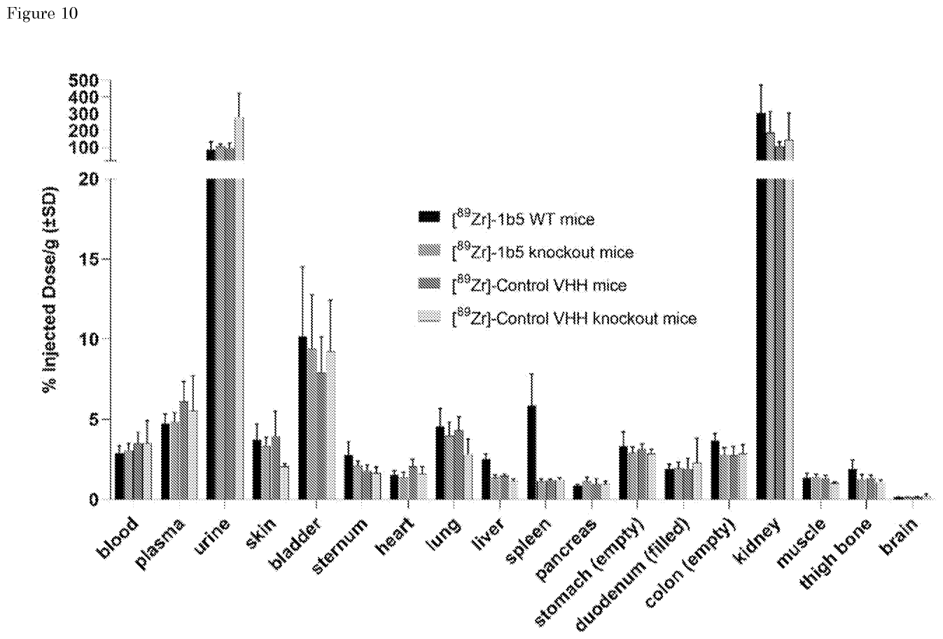

In vivo binding data of 1B5 is shown in FIG. 10. Specific binding of 1B5 is observed in spleen.

### Example 3 Adhesion Assay

### Materials and Methods

### Binding of HL-60 to Recombinant Human CD169

Recombinant human CD169 protein was coated on Nunc MaxiSorp ELISA plates (Thermo Fisher Scientific) at 4° C. overnight. This was followed by blocking with Carbo-free blocking buffer (Vector labs, SP-5040-125) for 30 minutes at 37° C. Next, incubation with VHHs (500 nM), or a commercial antibody control (clone 7-239) was performed for 30 minutes at room temperature. In the meantime, HL-60 cells were harvested and labeled with CellTrace Violet for 7 min at 37° C. in PBS followed by washing with 0.5% BSA/PBS. Cells were then added to wells (40,000 cells), centrifuged briefly at 300×g, and incubated for 30 minutes at 37° C. To removed unbound cells, gentle washing with 100 μl of 37° C. PBS was performed three times. Plate-bound cells were visualized and counted on Cytation 5 cell imaging.

### Results

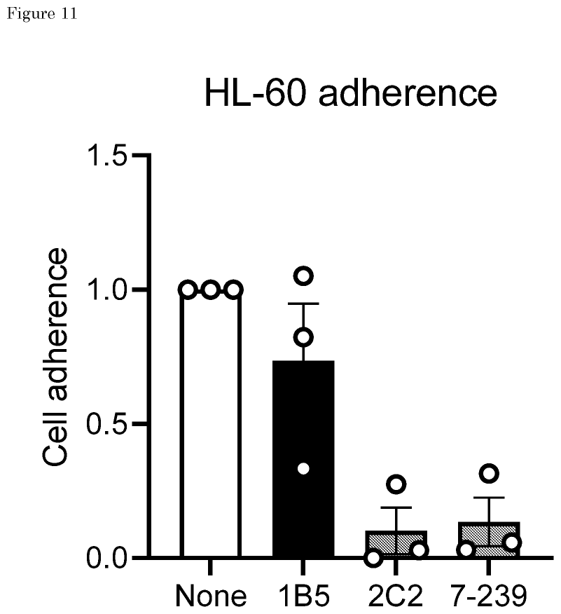

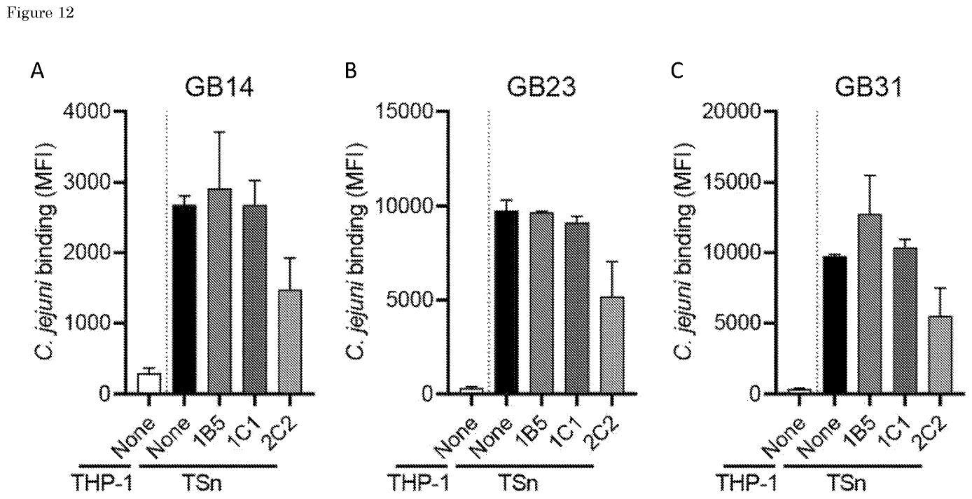

As is shown in FIG. 11, binding of the human leukemia cell line HL-60 cell line is inhibited by antibody 2C2 and control antibody 7-239, but not substantially by 1B5. This indicates that 1B5 does not interfere with CD169 ligand binding.

### Example 4 Pathogen Binding

### Materials and Methods

Binding of Campylobacter jejuni to THP-1 or TSn.

The human monocytic cell line THP-1 and human CD169-overexpressing THP-1 (termed TSn) cells were harvested, washed, and resuspended in RPMI-1640 media containing 1% FCS. Prior to binding, TSn was labeled with CellTrace Violet for 7 min at 37° C. in PBS followed by medium washing. Cells were pre-incubated with VHHs (500 nM) at 4° C. for 30 min. FITC-labeled C. jejuni was then added at a cell/bacterium ratio of 1:100 and incubated for 2 h at 37° C. in 5% CO₂ (Heikema et al., 2013. Infect Immun 81:2095-2103). The cells were washed to remove unbound bacteria and incubated with viability dye (Fixable viability dye eFluor 780, eBioscience; or Live Dead Blue, Life Technologies) for 10 min at 4° C. Cells were then fixed in 2% paraformaldehyde (PFA), and analyzed using Attune CytPix. Three different C. jejuni strains GB14, GB23, and GB31 were used.

Additionally, THP-1 and TSn cells were used to study transmission of human immunodeficiency virus (HIV) to the HIV sensitive cell line TZM-bl, as described (Sarzotti-Kelsoe et al., 2014. J Immunol Methods 409:131-146).

### Results

As is shown in FIG. 12A-C, binding of different C. jejuni strains to a monocyte cell line overexpressing human CD169 is inhibited by antibody 2C2, but not by antibodies 1B5 and 1C1. This indicates that 1B5 and 1C1 do not interfere with binding of C. jejuni strains to CD169, in contrast to 2C2.

As is shown in FIG. 12D, only antibody 2C2 blocks transmission of the HIV virus to a sensitive cell line, while 1B5, 1F6, 2F2, 1C1, and 1C9 hardly impede transmission. This indicates that 1B5, 1F6, 2F2, 1C1, and 1C9 do not interfere with binding of HIV to CD169, in contrast to 2C2.

### Example 5 In Vivo Immunization

### Materials and Methods

Liposomes were prepared from egg phosphatidylcholine, egg phosphatidylglycerol, cholesterol, molar ratio 3.8:1:2.5. 0.1 mol % DiD and 1% DSPE-PEG2000 maleimide was added. The organic phase was evaporated under reduced pressure using a rotavapor, after which 10 mM HEPES buffer (pH 6.4) containing 1 mg/ml OVA247-279 long peptide (sequence: DEVSGLEQLESIINFEKLTEWTSSNVMEERKIK, purified, produced in-house) was added for hydration. Samples were extruded through a 400/200 nm filter combination using high-pressure nitrogen (10-mL thermobarrel Lipex extruder; Northern Lipids, Burnaby, BC, Canada). Next, liposomes were separated from soluble peptide and concentrated to either 0.5 or 1 ml in 10 mM HEPES pH 7.4 by ultracentrifugation twice at 200,000 g (Beckman Coulter). The phosphate content of these liposomes was determined through an inorganic phosphate assay as described in Nijen Twilhaar et al., 2020 (Nijen Twilhaar et al., 2020. Pharmaceutics 12:1138).

CD169-targeting clone 1B5 and a control clone contained a C-terminal free thiol subsequently used for liposome conjugation via DSPE-PEG (2000)-maleimide. Prior to conjugation to DSPE-PEG (2000)-maleimide-containing liposomes, the antibodies were reduced using TCEP (Sigma Aldrich) at a molar ratio of 3:1 TCEP: antibody for 2 hours at room temperature (RT). Next, liposome suspensions were incubated at 4° C. overnight with the reduced antibodies at an antibody: DSPE-PEG (2000)-maleimide ratio of 1:40, 1:10 or 1:2 for liposome suspensions including 4 mol %, 1 mol % or 0.1 mol % DSPE-PEG (2000)-maleimide, respectively.

Subsequently, non-reacted maleimide groups were quenched using L-cysteine (Sigma Aldrich) in a ratio of 10:1 cys:DSPE-PEG (2000)-maleimide by incubation for 1 hour at RT. Non-conjugated antibodies and cysteine were removed by dialyzing against 600 mL 10 mM HEPES buffer pH 7.4 using Spectra-Por® Float-a-Lyzer® G2 100 kDa cutoff (Sigma Aldrich). The buffer was exchanged three times for every two hours with the last step being performed overnight. To increase the volume, 10 mM HEPES pH 7.4 buffer was then added up to 1 mL, after which the samples were centrifuged with Vivaspin 2 mL 100 kDa filters (Sigma Aldrich) for 5-10 minutes at 3000 g to reach 500 μl. Threreafter, 50 U/mL penicillin and 50 mg/mL streptomycin (Lonza, Basel, Switzerland) were added and samples were stored at 4° C.

Liposomes (total of 100 nmol in PBS; specific conditions indicated in respective figure legends) were supplemented with 25 μg of poly(I: C) (low molecular weight (LMW, InvivoGen, San Diego, CA, USA) and 25 μg of CD40-targeting antibody (clone 1C10, produced in house; equal to Creative Biolabs TAB-199LC) and injected in the tail vein of C57BL/6 mice. Spleens were harvested on day 7. Identification of antigen-specific CD8+ T cells was done using PE-labelled H-2Kb/OVA257-264 tetramer (LUMC, Leiden, The Netherlands), incubated for 45 minutes at 37° C. together with anti-CD8a antibody (clone 53-5.6, BD Biosciences, Franklin Lakes, NJ, USA). In addition, splenocytes were incubated with OVA257-264 and Golgiplug (BD Biosciences) for 5 hours, fixed using 2% paraformaldehyde (Electron Microscopy Sciences) and resuspended in 0.5% saponin to permeabilize the cell membrane. Subsequently, cells were stained with anti-IFNγ antibody (clone XMG1.2, eBioscience).

### Results

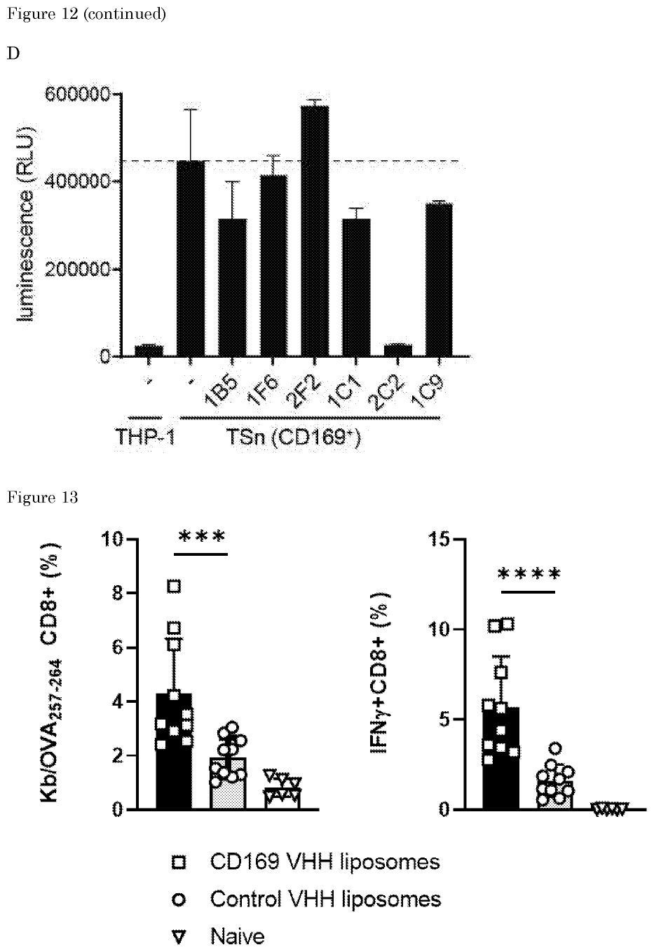

As is shown in FIG. 13, immunization with CD169 VHH (1B5) and OVA-containing liposomes resulted in increased OVA-specific CD8+ T cell priming.

### Example 6 Dendritic Cell Maturation

### Materials and Methods

CD169-specific antibody 1B5 with a free thiol was conjugated to CpG 1826 (InvivoGen) with a 3′ amine using the bifunctional linker succinimidyl 4-cyclohexane-1-carboxylate (SMCC). First, SMCC was conjugated to CpG 1826 and free SMCC was removed using PD-10 columns (GE Healthcare). Secondly, 1B5 was conjugated to CpG 1826 in a 1:1 ratio for 24 hours at 4° C. in the dark. The free CpG was subsequently removed with 4 successive washes in Amicon 10 kD centrifugal filters against PBS. Human CD169+ moDCs were pulsed with 500 nM stimuli for 45 minutes at 4° C., washed, and subsequently incubated for 16 hours at 37° C. and CD83 expression and IL-6 production were measured.

### Results

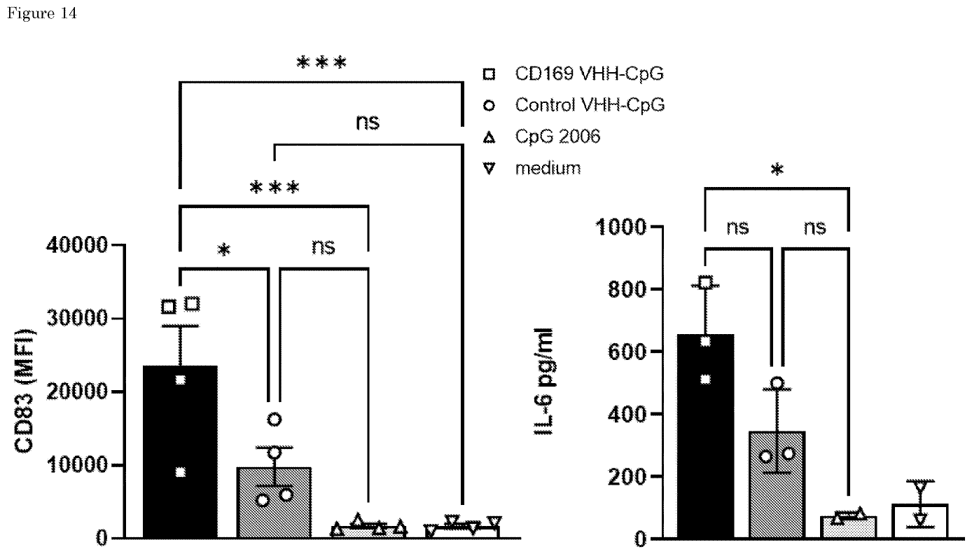

As is shown in FIG. 14, CpG linked to clone 1B5 significantly induces CD83 expression, when compared to CpG linked to a control antibody, or freed CpG. In addition, clone 1B5 induces more IL-6 production, when compared to the control antibody.

TABLE 1
CDR sequences of VHHs.

| | Family | | | |
|---|---|---|---|---|
| | Cluster | | | |
| Clone | ID | CDR1 | CDR2 | CDR3 |
| 1B5 | 27 | GRTFSNYT | INWSGERT | AQAFTSNTVGRSPANYQH |
| 1C1 | 58 | GNIFSINT | ITYAGST | NRKDWTMAGQGET |
| 1H9 | 79 | GSGFSSSA | IFSTGST | KISGSDY |
| 1H10 | 67 | GLAFSSYA | INSSGGST | AKDPWLLHSDS |
| 2B4 | 17 | GRTFSNYL | ISPSGGAT | AADGARRVWPGQNVHDYDD |
| 2B5 | 11 | GRTFSTYG | ISQSGGRI | AAQKTHSDSIVFTEELSYEN |
| 2C2 | 5 | GLTFSSYD | ISDRGGST | AAYGARYHSSYYSEAVDEYDY |
| 2C4 | 78 | GMLFSRAT | ISTGGLT | NTFDGFHP |
| 2C7 | 13 | GRTFGSYA | ISRAGVRT | AGKSASYDDSVYVAPGQFPF |
| 2F2 | 21 | GLTFSTYN | ISRTGSNT | AADRNLGTWPAKVAHEYDY |
| 2E1 | 40 | GRTENRYF | ITWSGGTT | AADPSSPNWPPVEYEH |
| 2G2 | 41 | GGTFSSYH | INWYGGAT | AARRGNFSWHVREYDY |
| 2H10 | 45 | GSFFSIHA | ITDGGTT | NIDIYTRRVGGFTAY |
| 3E3 | 81 | GIFFSNYV | IFSTGYT | NADTY |
| 4D2 | 1 | ERTFGSYA | ISWNGGLT | AADPNDYSGSYYYTSESALDSSRYAY |
| 4F2 | 65 | GSIGSINV | MRADSST | NVVSYSFGARSY |
| 4C9 | 43 | GIVFRIND | VSSGGST | KAAVQYYNIGSPSGV |

TABLE 2
LogEC50 values of VHHs based on binding to human
CD169-overexpressing BW-Sn cells at different
concentrations as measured by flow cytometry.

| Clone | LogEC50 |
|---|---|
| 1B5 | −9.4 |
| 2F2 | −8.2 |
| 1H10 | −7 |
| 1C1 | −6.9 |
| 2C4 | −6.8 |

TABLE 3
VHH sequences with framework (FW) regions and CDR regions indicated. The family cluster is indicated as well.

| | Family | | | | | | | |
|---|---|---|---|---|---|---|---|---|
| Clone | Cluster | FW1 | CDR1 | FW2 | CDR2 | FW3 | CDR3 | FW4 |
| 1B5 | 27 | EVQLVESGGGLVQAG | GRTF | MGWFRQAPGK | INWS | WYADFVKGRFTISRDNAKNTVHL | AQAFTSNTVGRSPANY | WGQGTQ |
| | | GSLRLSCSSS | SNYT | DSEFVSG | GERT | QMNSLNVEDTAVYFC | QH | VTVSS |
| 1C1 | 58 | EVQLVESGGGLVQAG | GNIF | MGWYRQAPGK | ITYA | NYADSVKGRFTISRDAKNTVSLE | NRKDWTMAGQGET | WGQGTQ |
| | | GSLRLSCAAS | SINT | QRDLVAL | GST | MNSLKVEDTAVYYC | | VTVSS |
| 1H9 | 79 | EVQLVESGGGLVQPG | GSGF | MYWYRQAPGK | IFST | RYAGSVKGRFAISRDNAKNTAYL | KISGSDY | WGQGTQ |
| | | GSLRLSCAAS | SSSA | QREFVAQ | GST | QMNSLKLEDTAVYYC | | VTVSS |
| 1H10 | 67 | EVQLVESGGGLVQPG | GLAF | MSWVRLAPGK | INSS | SYADSVKGRFTISRDNAKNTLYL | AKDPWLLHSDS | WGQGTQ |
| | | GSLRLSCAAS | SSYA | GPEWVST | GGST | QMNSLKPEDSAVYYC | | VTVSS |
| 2B4 | 17 | EVQLVESGGSLVQAG | GRTF | MGWFRQAPGK | ISPS | WYADSVRGRFTISRDNAKNTVYL | AADGARRVWPGQNVHD | WGQGTQ |
| | | GSLRLSCAAS | SNYL | ERDEVAA | GGAT | QMNSLKPEDTAVYYC | YDD | VTVSS |
| 2B5 | 11 | EVQLVESGGGLVQPG | GRTF | LGWFRQAPDK | ISQS | YYVDSVKGRFTISRDNAKNTVYL | AAQKTHSDSIVFTEEL | WGQGTQ |
| | | GSLRLSCAAS | STYG | EREFVSA | GGRI | QLNSLKPEDTAVYYC | SYEN | VTVSS |
| 2C4 | 78 | EVQLVESGGGLVQAG | GMLF | MSWFRQAPGK | ISTG | NYPDSVKGRFTISRDNAKNTVYL | NTFDGFHP | WGQGTQ |
| | | GSLRLSCVAS | SRAT | QRERVAS | GLT | QMNSLKPEDTAAYYC | | VTVSS |
| 2C7 | 13 | EVQLVESGGGLVQAG | GRTF | MAWFRQAPRK | ISRA | YYTDSVEGRETISRDNAKNTVYL | AGKSASYDDSVYVAPG | WGQGTQ |
| | | GSLRLSCAAS | GSYA | ERSVVAA | GVRT | QMNSLKPEDTAVYYC | QFPF | VTVSS |
| 2F2 | 21 | EVQLVESGGGLVQAG | STYN | EREFVAE | GSNT | QMNTLEPADMAVYTC | YDY | VTVSS |
| | | GSLRLSCAAS | GLTF | MAWFRQTPGK | ISRT | WYGDSAKGRFTISRDNAINTVYL | AADRNLGTWPAKVAHE | WGQGTQ |
| 2E1 | 40 | EVQLVESGGGLVQAG | GRTE | MAWFRQAPGK | ITWS | WYADSVKGRFTISRDNAKNTVYL | AADPSSPNWPPVEYEH | WGQGTQ |
| | | GSLRLSCAAS | NRYF | ERDFVAA | GGTT | QMNSLKSEDTAVYYC | | VTVSS |
| 2G2 | 41 | EVQLVESGGGLVQAG | GGTF | MGWFRQAPGK | INWY | WYADSVKGRFTISRDNAKNTVYL | AARRGNFSWHVREYDY | WGQGTQ |
| | | GSLRLSCAAS | SSYH | EREFVAD | GGAT | QINSLKPEDTANYYC | | VTVSS |
| 2H10 | 45 | EVQLVESGGGLVQPG | GSFF | MGWYRQAPGK | ITDG | NYADSVKGRFTISADNAKNTVYL | NIDIYTRRVGGFTAY | WGQGTQ |
| | | GSLRLSCAAS | SIHA | QRELVAA | GTT | QMDSLKPEDTAVYYC | | VTVSS |
| 3E3 | 81 | EVQLVESGGDLVQPG | GIFF | VTWYRQVPGK | IFST | RYDSSVKDRFTISRDNAKNTVYL | NADTY | WGQGTQ |
| | | GSLRLSCAAS | SNYV | QRDFVAQ | GYT | QMSSLKPEDTARYYC | | VTVSS |
| 4D2 | 1 | EVQLVESGGGLVQAG | ERTF | MGWFRQAPGK | ISWN | YYANSVLGRFTISRDNAKNTVFL | AADPNDYSGSYYYTSE | WGQGTQ |
| | | GSLRLSCAAS | GSYA | EREFVSA | GGLT | RMNSLKPEDTAVYYC | SALDSSRYAY | VTVSS |
| 4F2 | 65 | EVQLVESGGGLVQPG | GSIG | MGWYRQAPGK | MRAD | NYGDSVKDRFTISRDNAKNTLYL | NVVSYSFGARSY | WGQGTQ |
| | | GSLRLSCVAS | SINV | QRELVAI | SST | QTNNLKSEDTAVYYC | | VTVSS |
| 4C9 | 43 | EVQLVESGGGLVQAG | GIVE | MGWYRQAPGK | VSSG | NYADSVKGRFTISRDNGKNTVHL | KAAVQYYNIGSPSGV | WGQGTQ |
| | | NSLRLSCVAS | RIND | QRELVAT | GST | QMNNLKPEDTAVYYC | | VTVSS |
| 2C2 | 5 | EVQLVESGGGLVQAG | GLTE | MAWFRQAPGK | ISDR | AYADSVKGRFTISRDNAKNTVYV | AAYGARYHSSYY SEAV | WGQGTQ |
| | | GSLRLSCAAS | SSYD | ERVEVAA | GGST | QMNSLKPEDTAVYYC | DEYDY | VTVSS |

1. A single heavy chain variable domain antibody that specifically binds CD169, but does not impede normal functioning of CD169.

2. The single heavy chain variable domain antibody of claim 1 comprising complementarity-determining regions (CDRs) having amino acid sequences as depicted in Table 1.

3. The single heavy chain variable domain antibody of claim 1, that is complexed to one or more immunoreactive antigens.

4. The single heavy chain variable domain antibody of claim 3, wherein the one or more immunoreactive antigens are pathogenic antigens.

5. The single heavy chain variable domain antibody of claim 3, wherein the one or more immunoreactive antigens are viral and/or microbial antigens.

6. The single heavy chain variable domain antibody of claim 3, wherein the one or more immunoreactive antigens are tumor antigens.

7. The single heavy chain variable domain antibody of claim 3, wherein the one or more immunoreactive antigens are self-antigens or food or environmental allergens.

8. The single heavy chain variable domain antibody of claim 3, wherein the antibody is complexed to a carrier.

9. The single heavy chain variable domain antibody of claim 3, wherein the antibody is covalently bound to the one or more immunoreactive antigens.

10. A method of modulating an immune response in an individual, comprising administering the single heavy chain variable domain antibody according to claim 3 to the individual.

11. The method of claim 10, further comprising administering an immune modulating molecule to the individual.

12. An immunoreactive molecule comprising the single heavy chain variable domain antibody according to claim 3.

13. A pharmaceutical composition, comprising the single heavy chain variable domain antibody according to claim 3 and a pharmaceutically acceptable carrier.

14. A method of modulating an immune response in an individual against an immunoreactive antigen, comprising complexing the single heavy chain variable domain antibody according to claim 1 to one or more immunoreactive antigens to form an antibody-antigen complex and providing said antibody-antigen complex to the individual.

15. The method according to claim 14, wherein the antibody-antigen complex is provided to CD169 positive macrophages and/or dendritic cells of the individual.

16. The single heavy chain variable domain antibody of claim 1 wherein the normal functioning of CD169 comprises binding of CD169 to gangliosides.

17. The single heavy chain variable domain antibody of claim 8 wherein the carrier comprises a liposome comprising the one or more immunoreactive antigens.

18. The method of claim 11 wherein the immune modulating molecule comprises an adjuvant and/or a cytokine.

19. The method of claim 15 wherein the CD169 positive macrophages and/or dendritic cells comprise AXL receptor tyrosine kinase positive dendritic cells.


SEQUENCE LISTING (111 sequences)

SEQ ID NO: 1 - protein, 25 aa, synthetic construct
EVQLVESGGGLVQAGGSLRLSCSSS

SEQ ID NO: 2 - protein, 8 aa, synthetic construct
GRTFSNYT

SEQ ID NO: 3 - protein, 17 aa, synthetic construct
MGWFRQAPGKDSEFVSG

SEQ ID NO: 4 - protein, 8 aa, synthetic construct
INWSGERT

SEQ ID NO: 5 - protein, 38 aa, synthetic construct
WYADFVKGRFTISRDNAKNTVHLQMNSLNVEDTAVYFC

SEQ ID NO: 6 - protein, 18 aa, synthetic construct
AQAFTSNTVGRSPANYQH

SEQ ID NO: 7 - protein, 11 aa, synthetic construct
WGQGTQVTVSS

SEQ ID NO: 8 - protein, 25 aa, synthetic construct
EVQLVESGGGLVQAGGSLRLSCAAS

SEQ ID NO: 9 - protein, 8 aa, synthetic construct
GNIFSINT

SEQ ID NO: 10 - protein, 17 aa, synthetic construct
MGWYRQAPGKQRDLVAL

SEQ ID NO: 11 - protein, 7 aa, synthetic construct
ITYAGST

SEQ ID NO: 12 - protein, 37 aa, synthetic construct
NYADSVKGRFTISRDAKNTVSLEMNSLKVEDTAVYYC

SEQ ID NO: 13 - protein, 13 aa, synthetic construct
NRKDWTMAGQGET

SEQ ID NO: 14 - protein, 11 aa, synthetic construct
WGQGTQVTVSS

SEQ ID NO: 15 - protein, 25 aa, synthetic construct [sequence omitted]

SEQ ID NO: 16 - protein, 8 aa, synthetic construct
GSGFSSSA

SEQ ID NO: 17 - protein, 17 aa, synthetic construct
MYWYRQAPGKQREFVAQ

SEQ ID NO: 18 - protein, 7 aa, synthetic construct
IFSTGST

SEQ ID NO: 19 - protein, 38 aa, synthetic construct
RYAGSVKGRFAISRDNAKNTAYLQMNSLKLEDTAVYYC

SEQ ID NO: 20 - protein, 7 aa, synthetic construct
KISGSDY

SEQ ID NO: 21 - protein, 11 aa, synthetic construct
WGQGTQVTVSS

SEQ ID NO: 22 - protein, 8 aa, synthetic construct
GLAFSSYA

SEQ ID NO: 23 - protein, 17 aa, synthetic construct
MSWVRLAPGKGPEWVST

SEQ ID NO: 24 - protein, 8 aa, synthetic construct
INSSGGST

SEQ ID NO: 25 - protein, 38 aa, synthetic construct
SYADSVKGRFTISRDNAKNTLYLQMNSLKPEDSAVYYC

SEQ ID NO: 26 - protein, 11 aa, synthetic construct
AKDPWLLHSDS

SEQ ID NO: 27 - protein, 11 aa, synthetic construct
WGQGTQVTVSS

SEQ ID NO: 28 - protein, 25 aa, synthetic construct
EVQLVESGGSLVQAGGSLRLSCAAS

SEQ ID NO: 29 - protein, 8 aa, synthetic construct
GRTFSNYL

SEQ ID NO: 30 - protein, 17 aa, synthetic construct
MGWFRQAPGKERDFVAA

SEQ ID NO: 31 - protein, 8 aa, synthetic construct
ISPSGGAT

SEQ ID NO: 32 - protein, 38 aa, synthetic construct
WYADSVRGRFTISRDNAKNTVYLQMNSLKPEDTAVYYC

SEQ ID NO: 33 - protein, 19 aa, synthetic construct
AADGARRVWPGQNVHDYDD

SEQ ID NO: 34 - protein, 11 aa, synthetic construct
WGQGTQVTVSS

SEQ ID NO: 35 - protein, 8 aa, synthetic construct
GRTFSTYG

SEQ ID NO: 36 - protein, 17 aa, synthetic construct
LGWFRQAPDKEREFVSA

SEQ ID NO: 37 - protein, 8 aa, synthetic construct
ISQSGGRI

SEQ ID NO: 38 - protein, 38 aa, synthetic construct
YYVDSVKGRFTISRDNAKNTVYLQLNSLKPEDTAVYYC

SEQ ID NO: 39 - protein, 20 aa, synthetic construct
AAQKTHSDSIVFTEELSYEN

SEQ ID NO: 40 - protein, 11 aa, synthetic construct
WGQGTQVTVSS

SEQ ID NO: 41 - protein, 25 aa, synthetic construct
EVQLVESGGGLVQAGGSLRLSCVAS

SEQ ID NO: 42 - protein, 8 aa, synthetic construct
GMLFSRAT

SEQ ID NO: 43 - protein, 17 aa, synthetic construct
MSWFRQAPGKQRERVAS

SEQ ID NO: 44 - protein, 7 aa, synthetic construct
ISTGGLT

SEQ ID NO: 45 - protein, 38 aa, synthetic construct
NYPDSVKGRFTISRDNAKNTVYLQMNSLKPEDTAAYYC

SEQ ID NO: 46 - protein, 8 aa, synthetic construct
NTFDGFHP

SEQ ID NO: 47 - protein, 11 aa, synthetic construct
WGQGTQVTVSS

SEQ ID NO: 48 - protein, 8 aa, synthetic construct
GRTFGSYA

SEQ ID NO: 49 - protein, 17 aa, synthetic construct
MAWFRQAPRKERSVVAA

SEQ ID NO: 50 - protein, 8 aa, synthetic construct
ISRAGVRT

SEQ ID NO: 51 - protein, 38 aa, synthetic construct
YYTDSVEGRFTISRDNAKNTVYLQMNSLKPEDTAVYYC

SEQ ID NO: 52 - protein, 20 aa, synthetic construct
AGKSASYDDSVYVAPGQFPF

SEQ ID NO: 53 - protein, 11 aa, synthetic construct
WGQGTQVTVSS

SEQ ID NO: 54 - protein, 8 aa, synthetic construct
GLTFSTYN

SEQ ID NO: 55 - protein, 17 aa, synthetic construct
MAWFRQTPGKEREFVAE

SEQ ID NO: 56 - protein, 8 aa, synthetic construct
ISRTGSNT

SEQ ID NO: 57 - protein, 38 aa, synthetic construct
WYGDSAKGRFTISRDNAINTVYLQMNTLEPADMAVYTC

SEQ ID NO: 58 - protein, 19 aa, synthetic construct
AADRNLGTWPAKVAHEYDY

SEQ ID NO: 59 - protein, 11 aa, synthetic construct
WGQGTQVTVSS

SEQ ID NO: 60 - protein, 8 aa, synthetic construct
GRTENRYF

SEQ ID NO: 61 - protein, 17 aa, synthetic construct
MAWFRQAPGKERDFVAA

SEQ ID NO: 62 - protein, 8 aa, synthetic construct
ITWSGGTT

SEQ ID NO: 63 - protein, 38 aa, synthetic construct
WYADSVKGRFTISRDNAKNTVYLQMNSLKSEDTAVYYC

SEQ ID NO: 64 - protein, 16 aa, synthetic construct
AADPSSPNWPPVEYEH

SEQ ID NO: 65 - protein, 11 aa, synthetic construct
WGQGTQVTVSS

SEQ ID NO: 66 - protein, 8 aa, synthetic construct
GGTFSSYH

SEQ ID NO: 67 - protein, 17 aa, synthetic construct
MGWFRQAPGKEREFVAD

SEQ ID NO: 68 - protein, 8 aa, synthetic construct
INWYGGAT

SEQ ID NO: 69 - protein, 38 aa, synthetic construct
WYADSVKGRFTISRDNAKNTVYLQINSLKPEDTANYYC

SEQ ID NO: 70 - protein, 16 aa, synthetic construct
AARRGNFSWHVREYDY

SEQ ID NO: 71 - protein, 11 aa, synthetic construct
WGQGTQVTVSS

SEQ ID NO: 72 - protein, 8 aa, synthetic construct
GSFFSIHA

SEQ ID NO: 73 - protein, 17 aa, synthetic construct
MGWYRQAPGKQRELVAA

SEQ ID NO: 74 - protein, 7 aa, synthetic construct
ITDGGTT

SEQ ID NO: 75 - protein, 38 aa, synthetic construct
NYADSVKGRFTISADNAKNTVYLQMDSLKPEDTAVYYC

SEQ ID NO: 76 - protein, 15 aa, synthetic construct
NIDIYTRRVGGFTAY

SEQ ID NO: 77 - protein, 11 aa, synthetic construct
WGQGTQVTVSS

SEQ ID NO: 78 - protein, 25 aa, synthetic construct
EVQLVESGGDLVQPGGSLRLSCAAS

SEQ ID NO: 79 - protein, 8 aa, synthetic construct
GIFFSNYV

SEQ ID NO: 80 - protein, 17 aa, synthetic construct
VTWYRQVPGKQRDFVAQ

SEQ ID NO: 81 - protein, 7 aa, synthetic construct
IFSTGYT

SEQ ID NO: 82 - protein, 38 aa, synthetic construct
RYDSSVKDRFTISRDNAKNTVYLQMSSLKPEDTARYYC

SEQ ID NO: 83 - protein, 5 aa, synthetic construct
NADTY

SEQ ID NO: 84 - protein, 11 aa, synthetic construct
WGQGTQVTVSS

SEQ ID NO: 85 - protein, 8 aa, synthetic construct
ERTFGSYA

SEQ ID NO: 86 - protein, 17 aa, synthetic construct
MGWFRQAPGKEREFVSA

SEQ ID NO: 87 - protein, 8 aa, synthetic construct
ISWNGGLT

SEQ ID NO: 88 - protein, 38 aa, synthetic construct
YYANSVLGRFTISRDNAKNTVFLRMNSLKPEDTAVYYC

SEQ ID NO: 89 - protein, 26 aa, synthetic construct
AADPNDYSGSYYYTSESALDSSRYAY

SEQ ID NO: 90 - protein, 11 aa, synthetic construct
WGQGTQVTVSS

SEQ ID NO: 91 - protein, 25 aa, synthetic construct
EVQLVESGGGLVQPGGSLRLSCVAS

SEQ ID NO: 92 - protein, 8 aa, synthetic construct
GSIGSINV

SEQ ID NO: 93 - protein, 17 aa, synthetic construct
MGWYRQAPGKQRELVAI

SEQ ID NO: 94 - protein, 7 aa, synthetic construct
MRADSST

SEQ ID NO: 95 - protein, 38 aa, synthetic construct
NYGDSVKDRFTISRDNAKNTLYLQTNNLKSEDTAVYYC

SEQ ID NO: 96 - protein, 12 aa, synthetic construct
NVVSYSFGARSY

SEQ ID NO: 97 - protein, 11 aa, synthetic construct
WGQGTQVTVSS

SEQ ID NO: 98 - protein, 25 aa, synthetic construct
EVQLVESGGGLVQAGNSLRLSCVAS

SEQ ID NO: 99 - protein, 8 aa, synthetic construct
GIVFRIND

SEQ ID NO: 100 - protein, 17 aa, synthetic construct
MGWYRQAPGKQRELVAT

SEQ ID NO: 101 - protein, 7 aa, synthetic construct
VSSGGST

SEQ ID NO: 102 - protein, 38 aa, synthetic construct
NYADSVKGRFTISRDNGKNTVHLQMNNLKPEDTAVYYC

SEQ ID NO: 103 - protein, 15 aa, synthetic construct
KAAVQYYNIGSPSGV

SEQ ID NO: 104 - protein, 11 aa, synthetic construct
WGQGTQVTVSS

SEQ ID NO: 105 - protein, 28 aa, synthetic construct
VTHTYLEPGPVTANRQLYPEWTEAQRLD

SEQ ID NO: 106 - protein, 8 aa, synthetic construct
GLTFSSYD

SEQ ID NO: 107 - protein, 17 aa, synthetic construct
MAWFRQAPGKERVFVAA

SEQ ID NO: 108 - protein, 8 aa, synthetic construct
ISDRGGST

SEQ ID NO: 109 - protein, 38 aa, synthetic construct
AYADSVKGRFTISRDNAKNTVYVQMNSLKPEDTAVYYC

SEQ ID NO: 110 - protein, 21 aa, synthetic construct
AAYGARYHSSYYSEAVDEYDY

SEQ ID NO: 111 - protein, 33 aa, synthetic construct
DEVSGLEQLESIINFEKLTEWTSSNVMEERKIK